(19) United States
(12) Patent Application Publication
(10) Pub. No.: US 2022/0133530 A1
(43) Pub. Date: May 5, 2022

(54) Fidget Cold Pack

(71) Applicant: Fred Hsu, Port Washington, NY (US)

(72) Inventor: Fred Hsu, Port Washington, NY (US)

(21) Appl. No.: 17/577,154

(22) Filed: Jan. 17, 2022

(51) Int. Cl. A61F 7/10

(52) U.S. Cl. CPC A61F 7/106; A61F 2007/0036

(57) ABSTRACT

A cold pack that can be fidgeted in a human hand. The cold pack encases a heat-transferring substance and provides a rigid portion suitable for being securely held by and readily fidgeted with fingers of said human hand. Heat-conducting grooves and heat-insulating surfaces are built into the rigid portion, with acute edges, jutting bumps, guard rails and other textures to provide grip. Fingertips alternate between holding the cold pack by said grooves and surfaces, thereby applying dynamic cold therapy to the same fingertips, rest of fingers, the hand, and other body parts requiring treatment for swelling, inflammation and trauma arising from friction, compression or minor burn injuries. Timely application of dynamic cold therapy can thus prevent formation of blisters in fingers and hands commonly seen in string players, musicians, sportspeople and workers caused by repetitive contacts with objects, and by unexpected contacts with hot surfaces.

### TECHNICAL FIELD OF THE INVENTION

Various embodiments of the present disclosure relate to cold packs, and in particular to cold packs held by fingers in order to preempt the formation of blisters on fingertips resulting from playing string music instruments, from practicing certain sports, and from minor burn injuries.

### BACKGROUND OF THE INVENTION

Musicians that play string instruments are susceptible to finger blisters. Specifically, those who pluck at strings (harp, bass) or pin strings against a fingerboard (cello, guitar) may develop blisters on their fingertips and thumb tips, from both pressure and friction. These blisters start out only as a red sore on fingertips. Continued practices cause further swelling. Eventually plasma fluid starts to fill the space between skin and body, creating blisters. Even if a musician stops playing after developing red sores, blisters may still develop within hours.

Fingertip blisters are a major obstacle for beginners whose finger skins have not been subjected to chronic abuse by strings. Eventually, repeated blistering prompts the body to build up thick layers of callus on susceptible fingertip areas, making fingertip less of an issue for seasoned musicians. But even seasoned musicians suffer from occasional blistering.

Abundant materials can be found online which instruct musicians on dealing with blisters, including draining blisters, dressing a wound, and wrapping wounds. But few address the prevention of blister formation. The few that discuss preventions are limited to wrapping fingertips with a layer of cloth or rubber. But doing so distorts music making, and thus is not an ideal solution.

There exists a way to prevent blistering, without requiring wrapping fingertips with extra materials. This involves cooling red sores with a cold pack to alleviate the swelling and the pain. When applied to red sores on all fingertips before plasma fluid builds up, cold packs can prevent or reduce the formation of blisters. If a musician applies such cold therapy consistently after every practice, they will eventually build up enough calluses on these skin areas, without sustaining painful and harmful blisters. Once enough layers of calluses build up, the need for further cold therapy is greatly reduced.

However, there were no products designed for safe and dynamic application of cold therapy to these fingertip areas. Consequently, cold therapy for fingertip blisters was not known to string players, even thought it was prevalent in the medical field. Or perhaps it was the opposite. No one had recognized that such a well-known medical process can be applied to treat finger blisters in string players. And thus no practical products were created to facilitate this treatment. This unrecognized problem also applies outside string playing. For instance, percussionists are known to suffer finger blisters through friction against mallets. In various sports such as rowing, golfing and baseball, players are known to develop finger blisters. In a home kitchen, fingertip blisters are a result of common burn injuries. But no practical products existed to address these issues.

Rigid cold packs for lunch boxes can be used as a last resort to treat red sores. I have in fact used a simple, plastic cold pack made for lunch boxes for many years, to prevent fingertip blisters from forming when I occasionally play the cello. I would try to hold the cold pack with my fingers, using only red sore areas that require therapy. But it doesn't work well. I've always struggled to grip this slippery cold pack with my fingertips. It is also bulkier and heavier than needed, for cold therapy on small fingertip areas.

An effective cold therapy lasts for 15 to 30 minutes, with brief but intense cooling of sore areas, interrupted by intermittent breaks for fingertips to warm up before the next cooling phase. This dynamic form of cold therapy differs from a typical cold compress which applies static compression on an injured body part. I fidget the cold pack with fingertips that require treatment, squeezing select fingertips against cold surfaces as I see fit. While applying cold therapy on one hand, I would be walking about, tending to other businesses with my other hand at times, and with both hands when both hands are needed. I drop off the cold pack on a table, and pick it up as required. Or I pass it from one hand to the other. Thus, cooling devices such as gloves with freezer gel, finger sprints with freezer gel, freezer gel bags strapped to the palm, and similar flexible devices that wrap around fingers are not practical. They are designed to stay on a body part throughout the entire duration of the therapy. They provide constant temperature that are either too cold for too long, or too little and not effective. They do not lend themselves to being actively manipulated by fingers in alternating cycles of cold therapy and breaks.

While in an intermittent break, I may be carrying the cold pack in a hand, as I walk about. Conventional rigid cold packs are designed to transmit heat (and coldness) through all available surfaces for efficient cooling of food items. Therefore, there are no good ways to hold such rigid cold pack in a hand during these breaks—everywhere the hand and fingers touch are cold. They do not lend themselves to being actively manipulated by fingers in alternating cycles of cold therapy and breaks.

Let me summarize it: there aren't cold packs that can be fidgeted with fingers.

Conventional cold packs can be classified into two types. They are either rigid or flexible. In general, rigid cold packs are used inside a thermal container to keep food cold. They are designed to be fully heat-conducting in all surfaces for best results. Rigid cold packs are not meant to be fidgeted for longer than a few seconds. On the other hand, flexible cold packs are wrapped around either a beverage or a human body part. They are designed to be strapped to the intended target, and they stay in contact with said target for an extended period of time. Flexible cold packs are not meant to be fidgeted throughout a therapy session by human fingers. Their flexible shape also makes it hard for fingertips to grip them and fidget them.

In short, a string player's finger blister issue can be better addressed by an improved cold pack with a rigid component that fingers can securely hold and actively manipulate throughout a dynamic session of cold therapy. But rigid cold packs are traditionally designed to keep food items cold within a container, and are thus ill-suited for the needs of string players. The present disclosure improves upon traditional rigid cold packs, and transforms them into various fidgetable embodiments to serve newly-recognized needs.

### BRIEF SUMMARY

The background section revealed the previously unrecognized problem of the need to safely and dynamically apply cold therapy to sore fingertips caused by pressure and friction. Various embodiments of the present disclosure address this problem with a fidgetable product that can better apply cold therapy to prevent blisters from forming on fingertips due to said pressure and friction. In additional to addressing this specific problem domain, additional embodiments may be used to provide heat treatment instead of cold therapy to similar finger areas for different medical conditions. Yet further variations of these embodiments may change placement of heat-conducting and heat-insulating materials, and enable fingers to grip a pack for extended periods of time, to apply other surface areas of the pack to different body parts in order to treat swelling, inflammation and trauma.

The first embodiment of the present disclosure is shown in FIG. 1A and FIG. 1B. A rectangular thermal pack constructed from waterproof materials contains inside it a heat-transferring substance such as freezer gel, making it a cold pack. There are four finger-size recesses or grooves on the top face of this pack. There is a thumb-size recess or groove on the bottom face of this pack. These grooves are slightly slanted to allow a human hand to comfortably and securely grip the cold pack with four fingers and a thumb. The pack is roughly rigid in shape, thus won't deform and slip away from fingers. In this embodiment, grooved surfaces are constructed with a mildly heat-conducting material suitable for contact with human skin. Surrounding faces on the sides of the pack are constructed from a heat-insulating material, allowing fingers to hold these surfaces without feeling the coldness.

One application of the first embodiment is the prevention of blister formation on fingertips of a string player such as a cellist. For cellists, and especially for beginner cellists, blisters often develop on fingertips of the left hand, soon after a practice session where they receive sustained abuses in the form of concentrated high pressure and friction from metal strings. Fingertips are illustrated in FIG. 1G. Abovementioned susceptible fingertip areas are indicated in FIG. 1H. For advanced cellists, the thumb may also develop blisters when the thumb position is used for high notes beyond the neck on the fingerboard. The thumb tip and the susceptible thumb tip area are also illustrated in FIG. 1G and FIG. 1H.

After a practice session, a cello player simply takes this cold pack out of a freezer with the fingering hand which is usually the left hand. Only the four fingertips and the thumb tip are needed in order to grasp this cold pack securely in the left hand, as shown in FIG. 1Q and FIG. 1R. All finger surfaces fidgeting the cold pack are exactly where cold therapy is intended for. A brief 30 seconds of contact between fingertips and grooves provides some relief of pain and some reduction in the build-up of plasma fluid. Fingers can then move to an alternative gripping position, holding the cold pack via insulated surrounding faces instead, as shown in FIG. 1U. This allows fingertips to warm up again, and at the same time, grooves to be cooled down again by the freezer gel. 20-minute of cold therapy with alternating contacts and non-contacts with cold grooves thus preempts the formation of blisters.

This cold pack also works for musicians playing violins, violas, double basses, guitars, harps, etc. For some players, the right hand requires treatment as well, as these musicians pluck at strings with their right hand, and consequently may develop blisters on the right hand. Grooves are designed so they can be grasped from either side of the pack, as shown in FIG. 1X and FIG. 1Y. Thus, the same cold pack can be passed from the left hand to the right hand to treat fingertips on the right hand.

The second embodiment of the present disclosure is shown in FIG. 2A and FIG. 2B. This is similar to the first embodiment, at a glance. But it adds four finger-size recesses, or grooves on the bottom face, where the first embodiment has just one groove for the thumb. This embodiment can be used in the same way as the first embodiment, shown in FIG. 2Q and FIG. 2R, where left-hand fingers grip grooves on the top face. But it also allows left-hand fingers to grip grooves on the bottom face, as illustrated in FIG. 2S. The two rows of grooves each on an opposite face give users choices when deciding on the best way to treat sore fingers. For instance, as fingertips warm up grooves on one side, they can flip the cold pack, and use cold grooves on the other side to continue the cold therapy. When passing the pack from one hand to a second hand, fresh, cold grooves on the opposite side can be used to continue cold therapy on the second hand. Again, insulated surrounding faces can be gripped by fingers during a break, as shown in FIG. 2U.

The second embodiment also provides something extra in finger grooves, compared to plain-vanilla grooves found in the first embodiment. Compare the second embodiment in FIG. 2A and FIG. 2B to the first embodiment in FIG. 1A and FIG. 1B. The second embodiment furnishes each groove on the top face with a raised ball bump, making the groove-ball pair resemble a marble in a bell jar. This bump appears to get in the way of a thumb trying to fit into the groove, as illustrated in FIG. 2S. Similarly, each groove on the bottom face has guard rails that turn a groove into the shape of a spade. These guard rails appear to hinder the thumb's grip on the groove, as illustrated in FIG. 2R. Furthermore, grooves on the top face are cut relatively deep into the pack, with groove surfaces meeting edges on the top face at a near acute angle. These seeming obstacles actually enhance finger grips. They sink into finger surfaces, allowing fingers to gain a secure purchase on the cold pack. Without these obstacles, groove surfaces will instead need to be built with grippy materials with a high coefficient of friction, or be textured with raised ridges and indented valleys.

The third embodiment of the present disclosure is shown in FIG. 3A and FIG. 3B. This revises the first embodiment, by adding a second row of finger-size grooves on the far side of the top face. This embodiment doubles the volume of the cold pack as a result. It holds more freezer gel, and thus can provide a longer therapy session compared to the first embodiment. Grooves on the second row are mirror images of the first row, allowing fingers from both hands to collaborate in fidgeting the cold pack, and be treated at the same time, as illustrated in FIG. 3Q and FIG. 3R.

The fourth embodiment of the present disclosure is shown in FIG. 4A and FIG. 4B, nicknamed The Brick. This provides two rows of grooves on both the top face and the bottom face. Thus, there are 4 rows of grooves in total. This cold pack can be fidgeted by both hands in a variety of ways, one of which is illustrated in FIG. 4Q and FIG. 4R, showing the left thumb on the top face, and the right thumb on the bottom face. The fourth embodiment does not arrange the two rows of bell jar grooves and the two rows of spade grooves in mirror images, unlike the third embodiment. Instead, grooves in the fourth embodiment retain the same orientation in both rows. Compare the fourth embodiment in FIG. 4A to the third embodiment in FIG. 3B, for instance. The fourth embodiment gives a user the choice of gripping a spade groove by its head, or by its handle.

The fifth embodiment of the present disclosure is shown in FIG. 5A, FIG. 5B, FIG. 5C and FIG. 5E, nicknamed The Bar. This is a revision of the second embodiment, adding circular grooves on container faces surrounding the top face and the bottom face. In this embodiment, the circular grooves are heat-insulated. There are two insulated grooves on the front face, and four insulated grooves on the rear face. Thus, fingers can grip and fidget these insulated grooves when not in active cold therapy, or when transporting the pack from place to place, as illustrated in FIG. 5U and FIG. 5W. As in many embodiments, The Bar contains within its shell a heat-transferring substance such as freezer gel. As in many embodiments, fingertips and the thumb tip are treated simply by gripping cold grooves on the top face and the bottom face, as illustrated in FIG. 5X and FIG. 5Y. All container faces are heat-conducting and suitable for contact with human skin, such that any available unused flat surfaces on any container face can be used to apply cold therapy to body areas outside fingertips.

The sixth embodiment of the present disclosure is shown in FIG. 6A, nicknamed Tom. This is a revision of The Bar from the fifth embodiment. It offers the same benefits from The Bar, and at the same time adding more recesses or grooves on the remaining unfurnished faces, namely the end faces of the rectangular bar. These additional grooves are a cowcatcher groove on the right face, and a channel groove on the left face, as can be further observed in FIG. 6C and FIG. 6D. These grooves allow additional surfaces of the thumb to be treated with the cold pack, including the radial side of the thumb which is used by cello players in thumb positions. FIG. 6S and FIG. 6T illustrate one way to grip Tom to treat such thumb sore. The same grooves can be used to treat sides of other fingers as well. One peculiar deviation of Tom from other embodiments is how the hand secures this cold pack. Tom is primarily held fast by the caging action of the thumb against the pinky.

The seventh embodiment of the present disclosure is shown in FIG. 7A, nicknamed The Hat. This is another revision of The Bar from the fifth embodiment. This embodiment has an expanded volume, similar to The Brick, providing a longer-lasting therapy. The Hat does not provide a second row of grooves, however. The expanded portion is instead shaped with curves of various radii, and made with a mildly heat-conducting material suitable for contact with human skin. FIG. 7E and FIG. 7F illustrate additional views of The Hat.

The expanded portion of The Hat includes flat surfaces on the top face and the bottom face, as well as curved surfaces on surrounding faces. Thus, The Hat can be used to treat fingertips of one hand via grooves, and at the same time, another hand or another body part with the expanded portion, as shown in FIG. 7Q and FIG. 7R. The expanded portion does not hinder the use of the cold pack when fingers grasp grooves from the “crown” side of the hat, as shown in FIG. 7S. Alternatively, fingers may grasp the insulated grooves when merely holding the cold pack without treating these same fingers, as shown in FIG. 7T, or when grasping the cold pack to treat other body parts, as shown in FIG. 7U and FIG. 7V. Furthermore, concave sections of the expanded portion may be used to treat the radial side of the thumb and other fingers. FIG. 7T illustrates a cellist's thumb being treated after strings bite into the radial side of the thumb at thumb positions.

One aspect of the eighth embodiment of the present disclosure is shown in FIG. 8A. This is a revision of the first embodiment where four plain grooves are provided on both the top face and the bottom face. The cold pack is made of heat-conducting materials for groove surfaces and face surfaces. A removable overlay can be attached to the top face, to turn the grooves on the top face heat-insulated, and/or to customize the shape and tactile feedback of these grooves. Flexible shells that fit into grooves on the bottom face are provided, such that individual bottom grooves may be customized, and/or turned into heat-insulated contacts. An overlay is secured to the cold pack via a snap-on mechanism. And shells are attached with adhesives or hook-and-loop fasteners.

Another aspect of the eight embodiment is shown in FIG. 8E. In this variation of the same attachment theme, a plain rectangular bar serves as a base container, and individual groove plates can be attached to the container surface. These groove plates can be heat-conducting or heat-insulating, and can be furnished with different types of tactile feedback and gripping geometries. While the base container is rigid, groove plates may be made of flexible materials. The base container together with attached groove plates thus form a complete fidget cold pack.

The ninth embodiment of the present disclosure is shown in FIG. 9A and FIG. 9B, nicknamed Kidney. Four finger-size grooves are provided on a curved top face, matching the natural contour of fingertips in a relaxed hand posture as demonstrated in FIG. 1G. Fingers hold Kidney in the same way they hold The Bar and The Hat. This can be seen in FIG. 9Q and FIG. 9R, showing fingertips of a left hand being treated with Kidney. FIG. 9S shows a right hand fidgeting the same cold pack. And FIG. 9T illustrates the cellist's thumb issue being treated with one of the two thumb-size grooves on the bottom face.

Kidney shares many similar features found in The Hat. It has ample flat surfaces and curved surfaces. These surfaces are made with a mildly heat-conducting material suitable for contact with human skin, just like The Hat. Thus, Kidney can also be used by one hand to treat a second body part. But Kidney provides flat surfaces on its surrounding faces and curved surfaces on its top and bottom faces, unlike The Hat.

The tenth embodiment of the present disclosure is shown in FIG. 10A and FIG. 10B, nicknamed Rowboat. The top face of Rowboat is curved, and not flat like found in The Bar and Tom. This curved top face follows the inner contour the four fingers as they curl in to form a clenched fist, shown in FIG. 10S and FIG. 10T. In the tenth embodiment, the bottom face curves upward at the two ends of the Rowboat, covering surfaces that would otherwise be considered right face and left face in some other embodiments such as The Bar. These up-sweeping ends are similar to the bow and the stern of a boat, which continuations of the same hull and keel that run the whole length of the boat. There are two thumb-size grooves on the bottom face in Rowboat. But they are found respectively at the right curved end (the bow), and the left curved end (the stern) of the same bottom face (the keel). The thumb can be treated with either thumb groove. Due to the ergonomics of how human hands work, five finger grooves are provided on Rowboat, instead of the typical set of four grooves in most embodiments. A typical adult hand will need some space between the thumb groove and the index finger, when holding Rowboat.

Unlike with Tom, the pinky does not need to cap the opposite end face from the thumb. Thus, the Rowboat is not held primarily by the thumb and the pinky. Instead, the palm and the four fingers secure Rowboat in place. Again, refer FIG. 10S and FIG. 10T. This is useful for treating more than fingertips—the entire inside surface of the hand can be made to contact the cold pack, if needed. This embodiment can be used to treat blistering issues arising from the use of mallets by marimba players. They also help with blistering issues from sports injuries in baseball, golf, rowing, etc. In addition, minor burns on the hand is a common injury in kitchens. This embodiment is also helpful preventing blistering, when applied right after a minor incident.

All embodiments in the present disclosure can also be gripped by one hand, in order to treat the other hand, or other parts of the body, for issues involving swelling, inflammation and trauma. Variations can be made such that surrounding faces of a cold pack provide flexible surfaces that can be used as a cold compress on another body part. And finger grooves can be made heat-insulated wherever the cold pack is used this way. In addition, a thermal pack may contain gel or liquid suitable for being heated, to provide heat-based treatment. Instead of a reusable gel or liquid, multiple ingredients may be mixed on demand to provide an instant hot pack or cold pack via endothermic chemical reactions.

It should also be understood that advantages and desirable outcomes described in the present disclosure are not meant to be limiting in any way, as one skilled in the art will appreciate that other advantages may also be realized upon practicing the present disclosure.

In summary, traditional rigid food cooling packs and flexible body treatment packs are not practical for thermal-therapy needs described in the present disclosure, including but not limited to dynamic treatments of cold therapy for the prevention of fingertip blisters in musicians, finger blisters from sports and minor burns, etc. In contrast, the present disclosure and its various fidget embodiments address these needs, resulting in following desirable outcomes:
1. A user can securely grip a thermal pack with just fingertips and a thumb tip.
2. A user can actively fidget a pack in hand throughout a dynamic therapy session.
3. Fingertips, the hand, and any other surfaces of the body can be treated.
4. Fingers can take refuge when holding the pack outside of active treatment.
5. A pack can be shifted, flipped, rotated, and otherwise manipulated by a single hand.

### BRIEF DESCRIPTION OF THE DRAWINGS

FIG. 1A is a perspective view of the first embodiment of the present disclosure, showing a fidget cold pack with four finger-size grooves on its top face, and in a cutaway, a freezer gel encased within the cold pack.

FIG. 1B is another perspective view of the same first embodiment from in FIG. 1A, showing a single thumb-size groove on the fidget cold pack's bottom face, and in a cutaway, a freezer gel encased within the cold pack.

FIG. 1G is a view of the left hand, showing the thumb and four fingers in a relaxed posture.

FIG. 1H is a view of the left hand of a cellist, showing typical areas where friction blisters develop on fingers that have not yet built up a thick layer of callus.

FIG. 1Q is a view of a left hand holding the first embodiment from FIG. 1A using only fingertips and the thumb tip which require cold therapy.

FIG. 1R is another view of the same left hand from FIG. 1Q being treated with the first embodiment, now showing the thumb gripping the bottom groove.

FIG. 1U is a view of a left hand holding the first embodiment via insulated surrounding faces, during one of many intermittent breaks throughout a cold therapy session.

FIG. 1X is a view of a right hand holding the first embodiment from FIG. 1A using only fingertips and the thumb tip which require cold therapy.

FIG. 1Y is another view of the same right hand from FIG. 1X being treated with the first embodiment, now showing the thumb gripping the bottom groove.

FIG. 2A is a perspective view of the second embodiment, showing four grooves on its top face, each with a ball bump, making each groove resemble a bell jar with a marble inside.

FIG. 2B is another perspective view of the same second embodiment from in FIG. 2A, showing its bottom face with four grooves with guard rails, each resembling a spade.

FIG. 2Q is a view of a left hand holding the second embodiment from FIG. 2A using only fingertips and the thumb tip which require cold therapy.

FIG. 2R is another view of the same left hand from FIG. 2Q being treated with the second embodiment, now showing the thumb gripping a spade groove on the bottom face.

FIG. 2S is a view of a left hand gripping spade grooves on the bottom face with four fingers, and a bell jar groove on the top face with the thumb.

FIG. 2U is a view of a left hand holding the second embodiment via insulated surrounding faces, during one of many intermittent breaks throughout a cold therapy session.

FIG. 3A is a perspective view of the third embodiment, showing two rows of spade grooves on its top face arranged in a mirror image of each other.

FIG. 3B is another perspective view of the same third embodiment from in FIG. 3A, showing its bottom face with two spade grooves arranged in mirror image of each other.

FIG. 3Q is a view of two hands being treated at the same time with the same fidget cold pack from the third embodiment shown in FIG. 3A.

FIG. 3R is another view of the same two hands from FIG. 3Q being treated with the third embodiment, now showing both thumbs gripping own spade grooves on the bottom face.

FIG. 4A is a perspective view of the fourth embodiment, nicknamed The Brick, showing two rows of bell jar grooves on its top face where grooves have the same orientation.

FIG. 4B is another perspective view of The Brick from in FIG. 4A, showing its bottom face with two rows of spade grooves in the same orientation.

FIG. 4Q is a view of two hands being treated at the same time with The Brick from in FIG. 4A, with the left thumb gripping a bell jar groove on the top face.

FIG. 4R is another view of the same two hands from FIG. 4Q being treated with The Brick, with the right thumb gripping a spade groove on the bottom face.

FIG. 5A is a perspective front view of the fifth embodiment, nicknamed The Bar, which is a revision of the fidget cold pack from the second embodiment, with additional, insulated circular grooves on surrounding faces, two of such insulated grooves are visible on the front face. Freezer gel inside the cold pack is shown in a cutaway.

FIG. 5B is another perspective view of The Bar from in FIG. 5A, showing its bottom face with a row of spade grooves, and its front face with two insulated grooves. Freezer gel inside the cold pack is shown in a cutaway.

FIG. 5C is another perspective rear view of The Bar from FIG. 5A, showing four insulated circular grooves on the rear face.

FIG. 5E is the same perspective rear view of The Bar from FIG. 5C, without reference numerals, and with a cutaway showing a freezer gel encased within the cold pack.

FIG. 5U is a front view of a left hand holding The Bar via insulated grooves on surrounding faces with the thumb tip on an insulated groove on the front face, during one of many intermittent breaks throughout a cold therapy session.

FIG. 5W is a rear view of the same left hand from FIG. 5U holding The Bar, with four fingertips gripping the four insulated grooves on the rear face.

FIG. 5X is a view of a right hand holding The Bar from FIG. 5A using only fingertips and the thumb tip which require cold therapy.

FIG. 5Y is another view of the same right hand from FIG. 5X being treated with The Bar, now showing the thumb gripping a spade groove on the bottom face.

FIG. 6A is a perspective view of the sixth embodiment, nicknamed Tom, which is a revision of The Bar from FIG. 5C, with a cowcatcher groove on the right face and a channel groove on the left face.

FIG. 6C is another perspective view of Tom from FIG. 6A, showing bell jar grooves on the top face, and a deep-cut cowcatcher groove on the left face.

FIG. 6D is another perspective view of Tom from FIG. 6A, showing spade grooves on the bottom face, and a channel groove on one end face.

FIG. 6S is a view of a left hand holding Tom from FIG. 6C, primarily between the thumb and the pinky via grooves on the two end faces, with three fingers resting on insulated grooves on the rear face.

FIG. 6T is a view of the same left hand from FIG. 6S holding Tom from FIG. 6D, primarily between the thumb and the pinky via grooves on the two end faces, and three fingers resting on insulated grooves on the rear face.

FIG. 7A is a perspective view of the seventh embodiment, nicknamed The Hat, which is another revision of The Bar from FIG. 5A, with an expanded and curved portion made with mildly heat-conducting materials suitable for contact with human skin.

FIG. 7E is another perspective view of The Hat from FIG. 7A, showing bell jar grooves on its top face, and insulated circular grooves on the front face.

FIG. 7F is another perspective view of The Hat from FIG. 7A, showing spade grooves on its bottom face, and insulated circular grooves on a surrounding face.

FIG. 7Q is a view of a left hand holding The Hat from FIG. 7E to treat left fingertips with bell jar grooves on the top face, as well as the palm surface of a right hand with the expanded and curved portion of The Hat.

FIG. 7R is another view of the left hand treating left fingertips and right hand palm from FIG. 7Q, showing the bottom face with spade grooves.

FIG. 7S is a view of a left hand treating left fingertips with bell jar grooves on the top face of The Hat from FIG. 7E, with fingers gripping grooves from the “crown” side of the hat.

FIG. 7T is a view of a left hand holding The Hat via insulated grooves, during one of many intermittent breaks throughout a cold therapy session. If needed, the thumb can be pressed against a concave section of the expanded portion to treat the radial side of the thumb which can be a sore spot for cellists playing thumb positions.

FIG. 7U is a view of a right hand holding insulated grooves on The Hat from FIG. 7E to treat the palm surface of a left hand with the expanded and curved portion of The Hat.

FIG. 7V is another view of the right hand treating left hand palm from FIG. 7U, showing the bottom face with spade grooves.

FIG. 8A is a perspective view of a removable and heat-conducting/heat-insulating overlay with custom finger grooves capable of being snapped onto the top face of a basic fidget cold pack, and a set of heat-conducting/heat-insulating shells that can be attached to individual grooves on the bottom face. This is one aspect of the eighth embodiment.

FIG. 8E is a perspective view of individual heat-conducting/heat-insulating groove plates that can be attached to a plain rectangular container whereby together they form a complete fidget cold pack with four top grooves and one bottom grove. This is another aspect of the eighth embodiment.

FIG. 9A is a perspective view of the ninth embodiment, nicknamed Kidney, showing four finger-size grooves arranged on a curved top face, matching the natural contour of fingertips in a relaxed hand posture.

FIG. 9B is another perspective view of Kidney from FIG. 9A, showing two thumb-size grooves on the bottom face of Kidney.

FIG. 9Q is a view of a left hand holding Kidney from FIG. 9A to treat left fingertips with bell jar grooves on the curved top face.

FIG. 9R is another view of the left hand treating left fingertips from FIG. 9Q, showing the thumb on one of the two bottom grooves.

FIG. 9S is a view of a right hand holding Kidney from FIG. 9A to treat right fingertips with bell jar grooves on the curved top face.

FIG. 9T is a view of a left hand holding Kidney from FIG. 9B, showing how a cellist's thumb can be treated with one of two bottom grooves.

FIG. 10A is a perspective view of the tenth embodiment, nicknamed Rowboat, showing five finger grooves on a curved top face which follows the inner contour of four fingers as they curl into a clenched fist, and a bow groove on one end of a curved bottom face.

FIG. 10B is another perspective view of Rowboat from FIG. 10A, showing the front face, the curved bottom face, and a bow groove on one end of the curved bottom face.

FIG. 10S is a view of a left hand holding Rowboat from FIG. 10A between fingers and the palm, to treat left fingertips with saddle grooves on the curved top face, and the thumb with a bow groove on one end of the curved bottom face.

FIG. 10T is another view of the left hand holding Rowboat from FIG. 10S, showing how the palm and inside surface of the hand can be treated with a clenched fist grip.

### REFERENCE NUMERALS

It is noted that like parts are designated by like reference numerals throughout the accompanying drawings. A list of numbered parts is presented below:
20. Top face of a fidget cold pack
22. Bottom face of a fidget cold pack
31. Front face of a fidget cold pack
33. Rear face of a fidget cold pack
35. Right face of a fidget cold pack
37. Left face of a fidget cold pack
40. Plain finger groove on a top face
41. Cowcatcher groove with a V-shaped groove surface bordered by nose knob 59
42. Plain finger groove on a bottom face
43. Channel grove cut with a V-shaped cross section into a container face
44. Bell jar groove with acute boundary edges
45. Half pipe groove with a half pipe surface 75 and two marble bumps 56
46. Spade groove with guard rails 58
47. Bell jar-like groove on a bow section of cold pack 110, Rowboat
49. A groove with a saddle-like surface flanking front and rear sides of Rowboat
50. Groove boundary of a finger groove
51. Groove boundary of a bell jar groove 44
52. Groove surface of a plain finger groove
53. Groove surface of a bell jar groove 44
54. Nadir point of a finger groove
55. Nadir point of a bell jar groove
56. Marble bump used as grip aid
57. One of two catcher surfaces in a cowcatcher groove 41
58. Guard rails flanking one end of spade groove 46
59. Nose knob used as a grip aid in a cowcatcher groove 41
60. A row of finger grooves on the top face bordering the front face
62. A row of finger grooves on the top face bordering the rear face
64. A row of finger grooves on the bottom face bordering the front face
66. A row of finger grooves on the bottom face bordering the rear face
70. Curved surrounding surfaces of cold pack 170 in the shape of the crown of a hat
71. A flat, expansive surface on either the front face or the rear face of Kidney 190
72. Curved surrounding surfaces of cold pack 170 in the shape of the brim of a hat
73. Curved surface on either end (right face or left face) of cold pack 190, Kidney
74. A flat, expansive surface on top face 20 of cold pack 170, The Hat
75. Groove surface of half pipe groove 45
76. A concave nook of crown curvature 70 suitable for treatment for a thumb
77. A notch that fits the latch of a snapped-on face overlay such as face overlay 82
78. A pinky-size nook located between crown curvature 70 and brim curvature 72
80. A circular, heat-insulated groove
82. Removable face overlay that can be snapped onto a face of a cold pack
84. Groove shell that can be attached to a finger groove on a cold pack
86. Groove plate that can be attached to a face on base container 182
88. Heat-insulated hull surface of front face 31 and rear face 33 of Rowboat 110
90. Freezer gel encased within a fidget cold pack
91. Curved surface on either end of the curved deck of cold pack 110, Rowboat
92. Hemisphere bump on a bow groove 47
93. Groove surface of saddle groove 49
94. Nadir point of a bow groove 47
95. Groove surface of a bow groove 47
96. Hemisphere bump in the center of a saddle groove 49
97. A flat, expansive surface on the keel of curved bottom face 22 of Rowboat
98. Groove boundary of a bow groove 47
99. Groove boundary of a saddle groove 49
100. First embodiment of fidget cold pack with four top grooves and one bottom groove
110. Rowboat—Tenth embodiment with five saddle grooves and a hull-like bottom face
120. Second embodiment with four bell jar grooves and four spade grooves
130. Third embodiment with two rows of grooves on the top face
140. Fourth embodiment with two rows of grooves on both top face and bottom face
150. The Bar—fifth embodiment with insulated grooves on surrounding faces
160. Tom—sixth embodiment with a cowcatcher groove and a channel groove
170. The Hat—seventh embodiment with curved surrounding faces
180. Basic cold pack with four top grooves and four bottom grooves
182. Plain rectangular base container
190. Kidney—ninth embodiment with grooves on curved top face and bottom face
200. A human left hand
210. Thumb tip of left hand 200
211. Fingertip of the index finger of left hand 200
212. Fingertip of the middle finger of left hand 200
213. Fingertip of the ring finger of left hand 200
214. Fingertip of the pinky finger of left hand 200
220. Area on the thumb susceptible to blister development on a cellist's left hand
221. Area on the index fingertip susceptible to blisters on a cellist's left hand
222. Area on the middle fingertip susceptible to blister on a cellist's left hand
223. Area on the ring fingertip susceptible to blister on a cellist's left hand
224. Area on the pinky fingertip susceptible to blister on a cellist's left hand
300. A human right hand
310. Thumb tip of right hand 300

Further areas of applicability of the present disclosure will become apparent from the detailed description provided hereinafter. It should be understood that the detailed description and specific examples, while indicating some embodiments of the disclosure, are intended for purposes of illustration only and are not intended to limit the scope of the disclosure.

### DETAILED DESCRIPTION

### First Embodiment

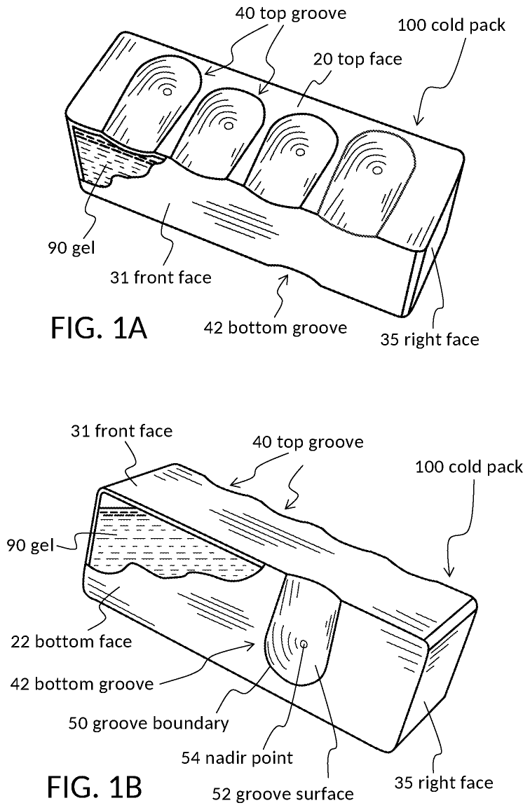

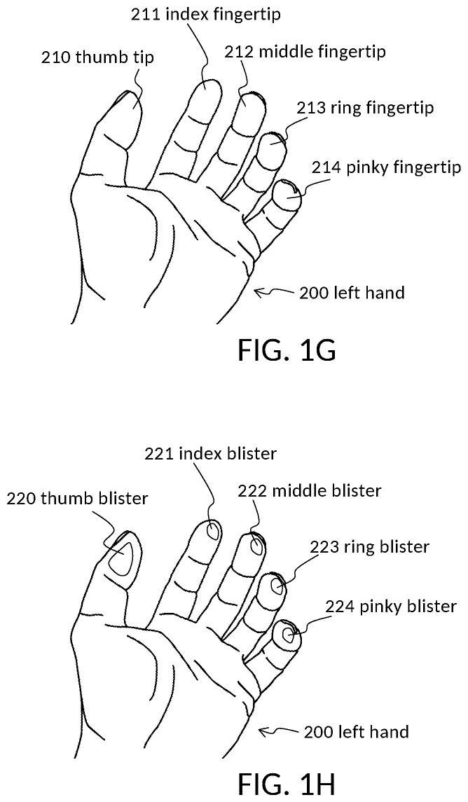

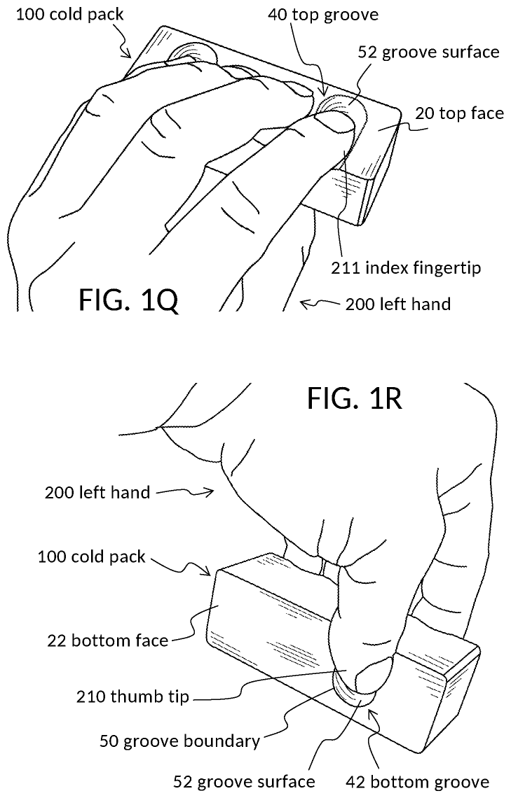

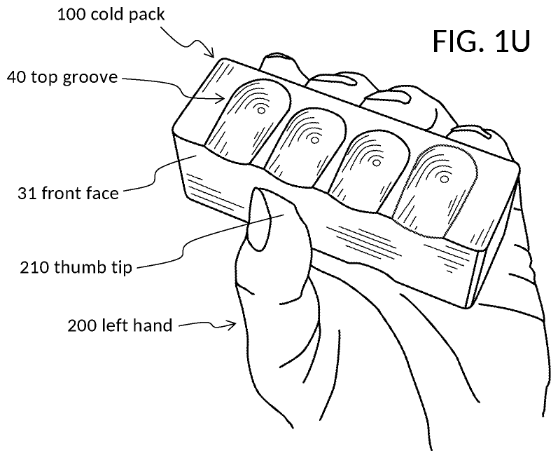

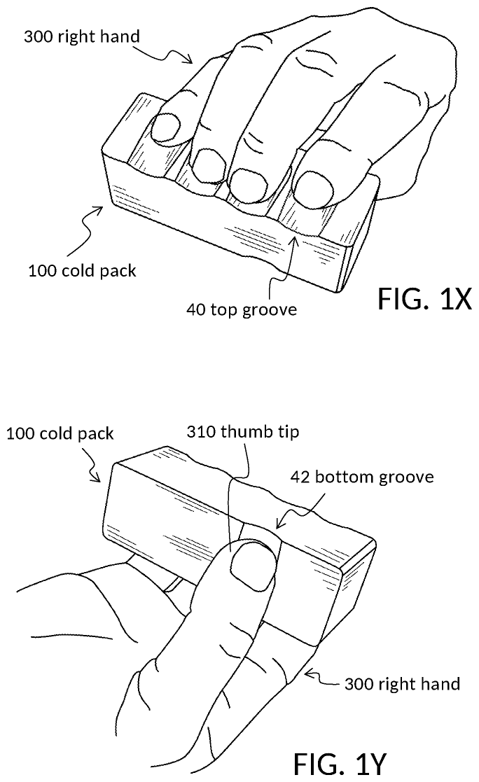

FIG. 1A illustrates the first embodiment of the present disclosure, showing a fidget cold pack 100 with four finger grooves on its top face 20. These finger grooves are thus called top grooves 40. In one aspect, the cold pack, or thermal pack, takes the form of a substantially rectangular bar acting as a container for a heat-transferring substance such as freezer gel 90 encased within. Top face 20 is one of many container faces that constitute the waterproof container shell of this cold pack. Two more container faces are visible in FIG. 1A, and they surround top face 20 from the front and from the right. They are named front face 31 and right face 35. An edge of a bottom groove 42 is visible where it intersects front face 31.

FIG. 1B illustrates the bottom face 22 of the first embodiment, where one bottom groove 42 is provided on bottom face 22 of cold pack 100 containing freezer gel 90. A groove boundary 50 demarcates the groove surface 52 of bottom groove 42 from bottom face 22. A nadir point 54 is the deepest part of groove surface 52. Front face 31 and right face 35 surround bottom face 22. Edges of top grooves 40 are visible where they intersect front face 31.

Both FIG. 1A and FIG. 1B illustrate some basic features of fidget cold packs. For instance, finger grooves are recesses that generally fit a fingertip or a thumb tip. Each groove generally has a groove boundary 50 that demarcates at a non-straight angle the generally-curved groove surface 52 from the container face that hosts the groove. A groove generally has an area of at least π*5 mm\hat{ }2. And the deepest nadir point 54 of a groove generally has a depth as measured from the average height of the boundary border greater than 3 mm. In one aspect, a basic fidget cold pack provides two or more top grooves 40 on top face 20, and one or more bottom grooves 42 on bottom face 22. This allows at least two of four fingers to grip the top face via top groove 40, and the thumb to grip the bottom face via bottom groove 42. In another aspect, grooves of a basic fidget cold pack are placed such that they are comfortably reachable by fingertips that contact them. For instance, in the first embodiment, top grooves are typically placed such that each is not more than 40 mm away from its nearest neighbor, with the distance being measured between nadir points of grooves.

In general, a cold pack's container shell can be divided into container faces with no strict requirements on their individual curvature and orientation. A flat container face is chosen as top face 20, and another one as bottom face 22 in FIG. 1A and FIG. 1B, which have already been described. The rest of container faces include surrounding faces, some of which are named, and others unnamed. Surrounding faces border top face 20 and bottom face 22. Some surrounding faces are named in this embodiment, such as front face 31. In many embodiments in the present disclosure, physical edges with non-straight surface angles exist as boundaries between faces. For instance, physical edges separate top face 20 from front face 31 in FIG. 1A. However, such geometrical features that segregate one face from another is not a requirement, but mere convenience. For instance, with a cold pack taking the shape of a perfect sphere, one skilled in the art will still be able to label different portions of its surface generally as a top face, a bottom face, a front face, a rear face, a right face, and a left face.

Furthermore, the set of container faces explicitly named and specified in the present disclosure together constitute a combined shape which should remain within a reasonable dimension such that a human hand can fidget the cold pack comfortably. In one aspect, the combined shape of specified container faces should fit within and should not exceed a sphere of 70 mm radius. In another aspect, the volume of said sphere of 70 mm radius can be used as an upper limit on the maximum volume or capacity of a practical fidget cold pack.

In general, both top face 20 and bottom face 22 are substantially rigid. And both faces are connected in a substantially rigid manner. This connection may be done inside the shell of the container, invisible to human inspections from the outside. Or the connection may be done via a surrounding face such as front face 31 in FIG. 1A and FIG. 1B. These rigid faces and their connections together form the substantially rigid portion of a cold pack. Top groove 40 and bottom grooves 42 are thus bound structurally to the rigid portion, and maintain their position and orientation with respect to one another, allowing a human hand to securely grip and fidget the cold pack, without the cold pack deforming or slipping away from fingers.

In general, the present disclosure applies to thermal packs containing a heat-transferring substance. Whether said substance is best used heated, cold, or both is irrelevant to the basic principles behind a fidgetable thermal pack. This disclosure focuses on describing use cases of cold therapy such as the first embodiment shown in FIG. 1A and FIG. 1B, using a freezer gel 90, with the understanding that one skilled in the art will appreciate how to apply the same principals to use cases of hot therapy. Said heat-transferring substance may be a gel or a liquid that can be heated or chilled, and thus making the thermal pack reusable. Said heat-transferring substance maybe also be created on demand from an endothermic chemical reaction of a plurality of ingredients which generate instantaneous temperature changes, but are generally for one-time use only. Maximum volume of said gel or liquid is limited by the practical container capacity discussed earlier.

In general, finger grooves and container faces may be constructed from the same material, or different materials. These materials may be heat-insulating or heat-conducting. They may be low-friction and smooth to the touch, or grippy and amenable to being gripped by fingers. Some may present a textured surface as an alternative in order to increase the coefficient of friction. Some container faces may be constructed from flexible materials. These material choices enable different applications of grooves and faces. In the first embodiment shown in FIG. 1A and FIG. 1B, grooved surfaces in top grooves 40 and bottom groove 42 are constructed with a mildly heat-conducting material suitable for contact with human skin during cold therapy treatments. Some surrounding faces such as front face 31 are constructed from a heat-insulating material, allowing fingers to hold these insulated surfaces without feeling the coldness when not in treatment.

FIG. 1G illustrates a human left hand 200, showing the thumb and four fingers in a relaxed posture. While the entire hand is susceptible to swelling, inflammation and trauma arising from friction, compression or minor burn injuries, the tip segments of the thumb and four fingers are especially susceptible to formation of blisters, because these are deployed in everyday manipulation of objects. Thumb tip 210, index fingertip 211, middle fingertip 212, ring fingertip 213 and pinky fingertip 214 are especially prone to being abused in everyday activities of string players, musicians, sportspeople and workers. Blisters develop after repetitive contacts with objects, and after unexpected contacts with hot surfaces from these everyday activities.

FIG. 1H illustrates a left hand 200 of a beginner cellist, showing the thumb and four fingers in a relaxed posture. Five areas of the hand are highlighted as highly susceptible areas for development of blisters, as a beginner cellist learns to play the cello before she has built up a thick layer of callus in her fingers. They are thumb blister area 220, index blister area 221, middle blister area 222, ring blister area 223 and pinky blister area 224. Thumb blister area 220 is caused by a cellist playing in the thumb position. The rest of blister areas result from fingertips being repeatedly used to pin one of the four cello strings against the fingerboard in all positions. Whereas blister areas on the four fingertips are located squarely on fingertips, thumb blister area 220 lays half way between the flesh of tip segment of the thumb, and the radial side of the thumb, also known as the lateral side of the thumb.

FIG. 1Q illustrates the operation of the first embodiment. Susceptible finger areas are treated with the first embodiment, by simply having left hand 200 pick up cold pack 100. The four fingertips touch cold groove surfaces as they grip and fidget cold pack 100. For instance, index fingertip 211 is shown touching cold groove surface 52 of one of four top grooves 40 on top face 20. Finger surfaces that fidget the cold pack are the exact target surfaces the cold therapy is intended for. A user thus applies cold therapy to fingertips to relief pain and to reduce plasma fluid build-up.

FIG. 1R continues the illustration of the operation of the first embodiment. It is another view of the same left hand 200 holding cold pack 100, now showing thumb tip 210 gripping bottom groove 42 on bottom face 22. It is noted that a cold pack filled with freezer gel presents an appreciable weight. The pack is therefore provided with features to help fingers grip it. Groove boundary 50 joins two surfaces at a non-straight angle, and serves as a gripping aid for finger skins. These two surfaces are groove surface 52 and bottom face 22. In addition, finger grooves are anchored on a substantially rigid portion of the pack, and thus are not squeezed out of place when pinched between fingers on the top face and the thumb on the bottom face. These features allow fingers to hold and fidget the cold pack without the pack slipping out of the hand. The basic design with multiple top grooves on the top face, and at least one bottom groove on the bottom face, thus accommodates the ergonomics of human hands with opposable thumbs.

FIG. 1U illustrate the nature of the dynamic cold therapy where fingers and the thumb take refuge during intermittent breaks between intense but short cold treatments to fingertips. FIG. 1U also illustrate again how human hands hold and grip objects with opposable thumbs. Previously in FIG. 1Q and FIG. 1R, fingers and the thumb are treated with cold therapy by simply gripping cold finger grooves. Now in FIG. 1U, left hand 200 instead grips surrounding faces constructed with a heat-insulated and grippy material. For instance, thumb tip 210 is seen holding cold pack 100 by front face 31 which is heat-insulated in this embodiment. A user thus allows fingers and the thumb to take shelter on insulated surrounding faces, for a break from the coldness. Fingers and the thumb warm up again during the break. And finger grooves such as top groove 40 can regain their low temperature from the freezer gel within the pack.

While a cellist's left hand is used as an example to illustrate many applications of various embodiments of the present disclosure, one skilled in the art will understand that similar injuries apply to other string players such as double bass players and guitar players. One skilled in the art will also know that similar injuries applies to both the left and the right hand of musicians such as harpists and guitarists who pluck strings. Sportspeople sustain similar blisters in both hands from prolonged rowing, golfing, ball-throwing, and bat-swinging. Workers who grip tools all day long suffer from similar injuries in both hands. Furthermore, cooks in a kitchen often unexpected touch hot objects, which results in blisters in both hands from minor burns.

FIG. 1X and FIG. 1Y illustrate how the fidget cold pack can be used by the right hand as well. To this end, grooves are designed so they can be grasped from either side of the pack. Thus, the same cold pack can be passed from the left hand to the right hand to treat fingertips on the right hand. FIG. 1X shows a right hand 300 holding cold pack 100, with four fingertips gripping top grooves 40. FIG. 1Y shows the thumb tip 310 of right hand 300 gripping bottom groove 42 of cold pack 100.

### Second Embodiment

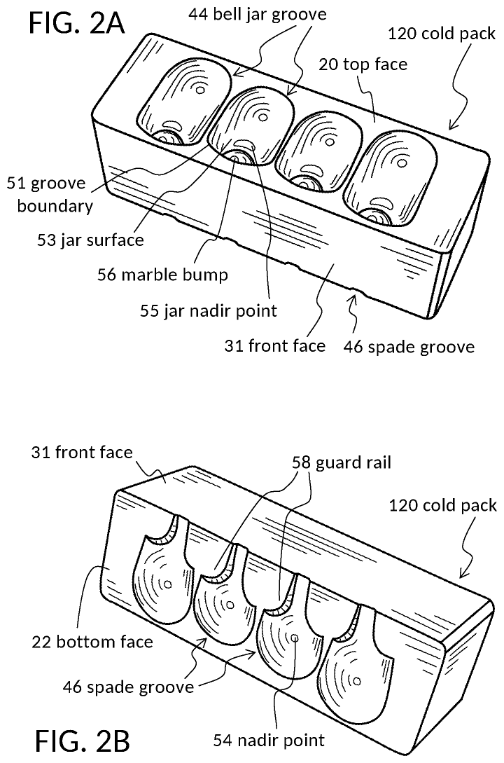

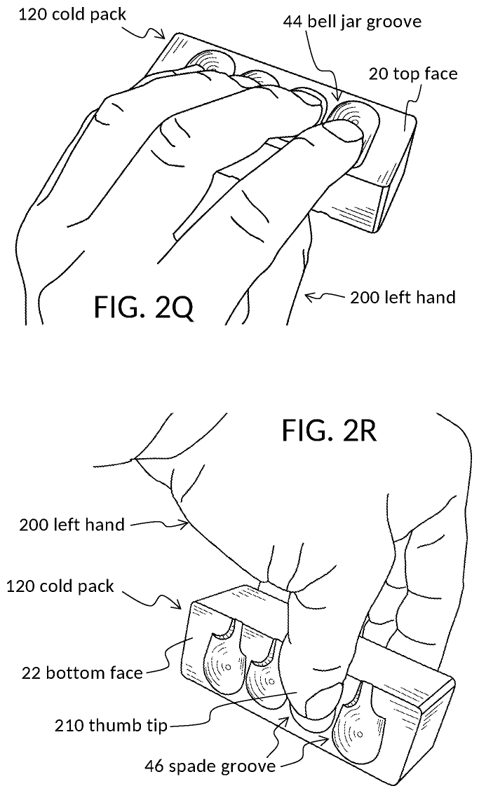

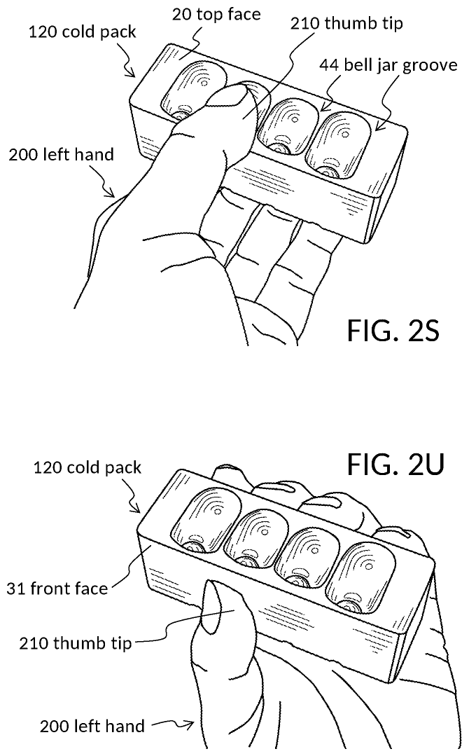

FIG. 2A illustrates the second embodiment of the present disclosure, showing a fidget cold pack 120 with four top grooves on its top face 20. These grooves are different from those in the first embodiment. The second embodiment features oval grooves on the top face that are carved further into the surface of top face 20 than found in the first embodiment, with a deeper bell jar nadir point 55. These oval grooves resemble a bell jar in shape, and are thus named bell jar grooves 44. The groove boundary 51 of a bell jar groove 44 features on average a near acute angle where top face 20 meets bell jar surface 53. On the end closer to front face 31, a marble-shaped bump 56 protrudes from the body of the pack into a bell jar groove 44.

FIG. 2B illustrates the bottom face 22 of the second embodiment, cold pack 120, where four bottom grooves with guard rails are provided. Bottom grooves on bottom face 22 are derived from bottom grooves 42 of cold pack 100—they have similar circular shape on the rounded end, and have the same nadir point 54. However, Bottom grooves on bottom face 22 are flanked by guardrail 58 on both sides of its non-rounded end, making each bottom groove take the shape of a spade. Thus these grooves are named spade grooves 46.

FIG. 2A and FIG. 2B also illustrate the concept of a row of finger grooves. The four bell jar grooves 44 on top face 20 in FIG. 2A form a row of grooves. In this embodiment, individual bell jar grooves 44 in the row line up close to the border between top face 20 and front face 31, roughly in the form of a mild curve. In general, however, any curve shape may be used, including a straight line which is a flat curve. Similarly, the four spade grooves 46 on bottom face 22 in FIG. 2B form a row of grooves, and line up close to the border between bottom face 22 and front face 31. Edges of the four spade grooves 46 can be seen in FIG. 2A where they intersect front face 31. In general, the nadir point of each groove in a row should be located no more than 30 mm away from said border. This allows fingers to grip these grooves comfortably, without forcing the purlicue of the hand to come into unintentional contacts with the cold pack.

FIG. 2Q and FIG. 2R illustrate the operation of the second embodiment. The second embodiment can be used in the same way as the first embodiment, previously shown in FIG. 1Q and 1R. That is, susceptible finger areas are treated in FIG. 2Q by simply having left hand 200 pick up cold pack 120. The four fingertips touch cold surfaces of a top row of bell jar grooves 44 on top face 20, as they grip and fidget cold pack 120. Similarly, thumb tip 210 of left hand 200 in FIG. 2R grips one of four spade grooves 46 on bottom face 22 of cold pack 120. In theory, thumb tip 210 can grip any of the four spade grooves 46. In reality, personal preferences and actual medical conditions will determine which groove a user actually uses.

FIG. 2S illustrates the operation of the second embodiment, where the four spade grooves on the bottom face are gripped by the four fingers, in contrast with the previous use case in FIG. 2Q where the four fingers grip grooves on the top face. Instead, in FIG. 2S, thumb tip 210 of left hand 200 grips one of four bell jar grooves 44 on top face 20 of cold pack 120.

FIG. 2U illustrates the operation of the cold pack 120 by left hand 200 via insulated surrounding faces, during one of many intermittent breaks throughout a cold therapy session. Thumb tip 210 is shown holding the heat-insulated front face 31 in this embodiment.

The addition of an entire bottom row of spade grooves 46 on bottom face 22 shown in FIG. 2R gives users choices when deciding on the best way to treat sore fingers. A user can alternate between gripping the top row and the bottom row, as these groove surfaces lose coldness to fingertips, and later regain coldness from the freezer gel. When passing the pack from one hand to a second hand, fresh, cold grooves on the opposite side can be used to continue cold therapy on the second hand.

The previous section on the first embodiment has already explained the importance that groove boundaries play. That is, a boundary with a non-straight edge angle helps fingers grip a cold pack. This same principle is expanded by bell jar grooves in the second embodiment. Specifically, bell jar grooves 44 shown in FIG. 2A are cut relatively deep into cold pack 120, with bell jar surfaces 53 meeting edges on top face 20 at a near acute angle. In general, a finger that grips a bell jar groove will feel at least two such acute edges sink into finger skin, as can be seen in FIG. 2Q and FIG. 2S, assuming that an oval boundary of a bell jar groove is divided into four edges: north, east, south and west. Furthermore, a bell jar groove has its nadir point at one end of its oval shape, accompanied by a ball bump jutting out of its groove surface into the space that a finger would otherwise occupy. This bump appears to get in the way of a thumb trying to fit into the groove, as shown in FIG. 2Q and FIG. 2S. This seeming obstacle actually increases grip needed for a good purchase, while reducing the area of skin required for contact. With the aid of adjacent edges, the ball bump allows a finger to literally stand on its fingertip, to confine cold therapy to this tip area which is a key susceptible finger area to blistering for string players, harpists, guitarists, and many more.

Similarly, spade grooves 46 shown in FIG. 2B enhances grip with guard rails 58. These seemingly get in the way of a finger trying to cram into all available groove space. But in fact these rails provide excellent gripping points to finger areas not actively seeking treatment, as illustrated in FIG. 2R. Without acute edges, jutting bumps, guard rails, or other forms of assistance, groove surfaces will instead need to be built with grippy materials with a high coefficient of friction, or be textured with raised ridges and indented valleys.

### Third Embodiment

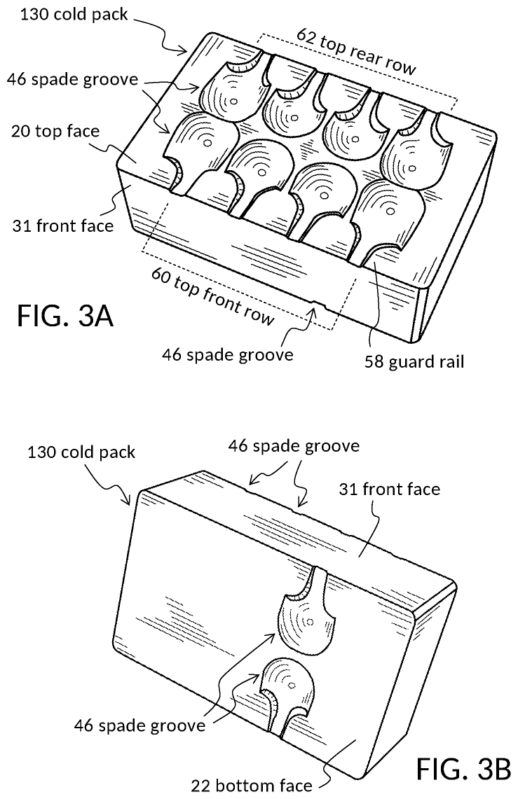

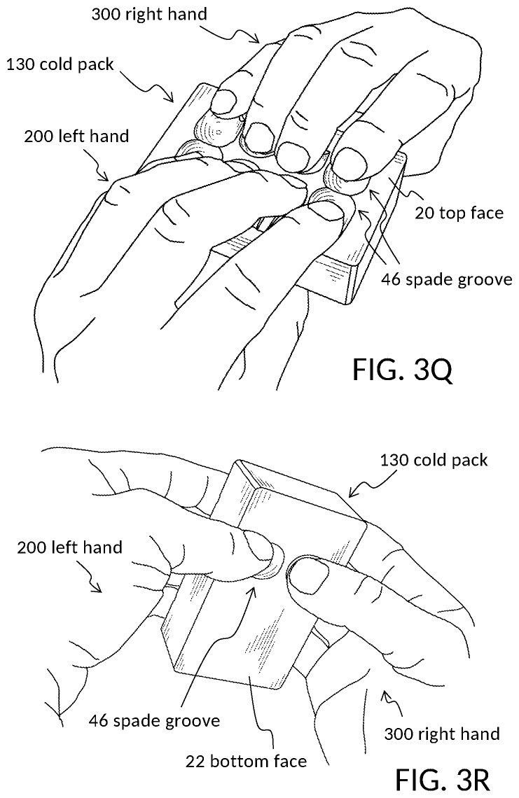

FIG. 3A illustrates the third embodiment of the present disclosure, showing a fidget cold pack 130 with two rows of spade grooves 46 on its top face 20, arranged in a mirror image of each other, with guard rails 58 of spade grooves pointing outward. The first row comprises four bell jar “row grooves”, borders front face 31 as its “border face”, and is labeled top front row 60. The second row borders the rear face of the cold pack, and is labeled top rear row 62. An edge of a spade groove 46 on the bottom face is visible where it intersects front face 31. The third embodiment almost doubles the volume of the cold pack, compared to the first two embodiments. It holds more freezer gel, and thus can provide a longer therapy session.

FIG. 3B illustrates the bottom face 22 of the cold pack 130, where two spade grooves 46 are provided. These two grooves are meant to be gripped by thumbs. And they are arranged in mirror image of each other. Edges of four spade groove 46 on the top face are visible where they intersect front face 31.

FIG. 3Q illustrates the operation of the third embodiment. A user treats susceptible finger areas of two hands simultaneously with the third embodiment, simply by having both left hand 200 and right hand 300 grip cold pack 130 with their fingers and their thumbs. Two rows each with four spade grooves 46 are visible on top face 20.

FIG. 3R continues the illustration the operation of the third embodiment. It is another view of the same left hand 200 and right hand 300 holding cold pack 130, now showing their respective thumbs gripping spade grooves 46 on bottom face 22.

### Fourth Embodiment—the Brick

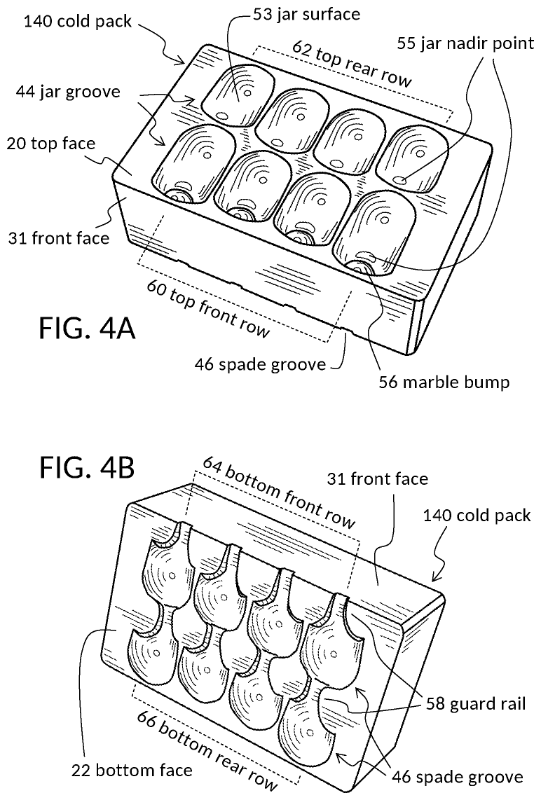

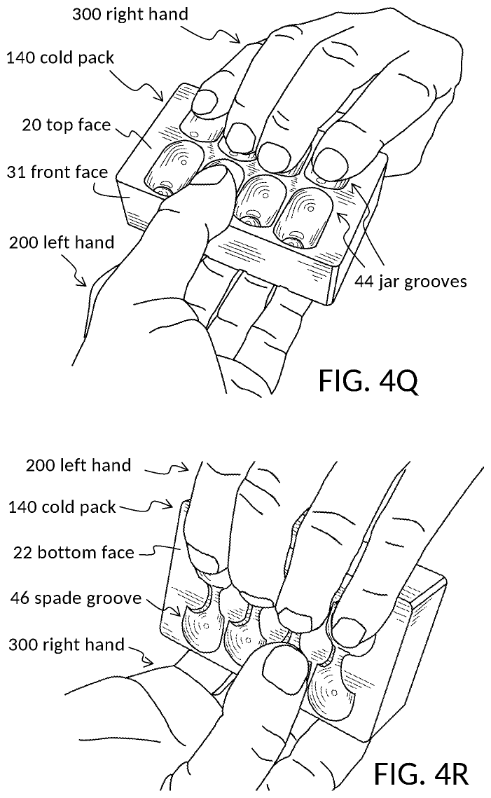

FIG. 4A illustrates the fourth embodiment of the present disclosure, nicknamed The Brick, showing a fidget cold pack 140, with two rows of bell jar grooves 44 on its top face 20, arranged in the same orientation. The first row borders front face 31, and is labeled top front row 60. The second row borders the rear face of the cold pack, and is labeled top rear row 62. Grooves in top front row 60 are provided each with a marble bump 56 next to its bell jar nadir point 55, to assist with gripping where fingers reach the groove from front face 31. Grooves in top rear row 62 do not need a marble bump, as they are reached by fingers from the rear face where jar surface 53 is shallower, compared to the nadir point 55. Edges of four spade groove 46 on the bottom face are visible where they intersect front face 31.

FIG. 4B illustrates the bottom face 22 of the cold pack 140, showing two rows of spade grooves 46 on bottom face 22, arranged in the same orientation. The first row borders front face 31, and is labeled bottom front row 64. The second row borders the rear face of the cold pack, and is labeled bottom rear row 66. Since grooves in both rows are arranged in the same orientation, all guard rails 58 point in the same direction, towards front face 31.

The Brick provides two rows of grooves on both its top face and its bottom face. Thus, there are 4 rows of grooves in total. This cold pack can be fidgeted by both hands in a variety of ways, one of which is illustrated in FIG. 4Q and FIG. 4R, showing the left thumb on the top face, and the right thumb on the bottom face.

FIG. 4Q illustrates the operation of the fourth embodiment, The Brick. Left hand 200 and right hand 300 are treated at the same time with cold pack 140, with the left thumb gripping a bell jar groove 44 with a marble bump on top face 20, and four fingers from right hand 300 gripping four bell jar groove 44 without a marble bump. As described earlier in the disclosure, marble bumps allow fingers to stand on their fingertips while still retaining a good grip on a groove. That is how the left thumb approaching from front face 31 grips bell jar groove 44. The four fingers from right hand 300, however, approach from the rear face, and thus lay flatter on the groove surface, with a larger contact interface for a wider treatment coverage.

FIG. 4R continues the illustration the operation of The Brick. It is another view of the same left hand 200 and right hand 300 holding cold pack 140, now showing the right thumb gripping the head of a spade groove 46 on bottom face 22, and four fingers from left hand 200 gripping handles of spade grooves 46.

### Fifth Embodiment—the Bar

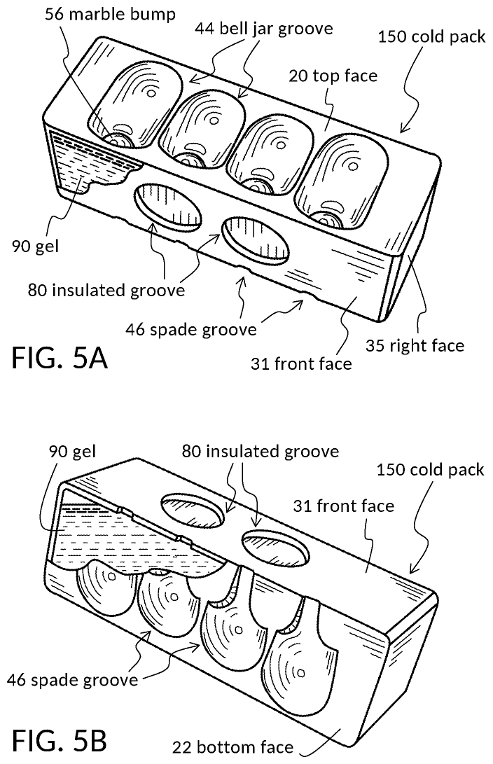

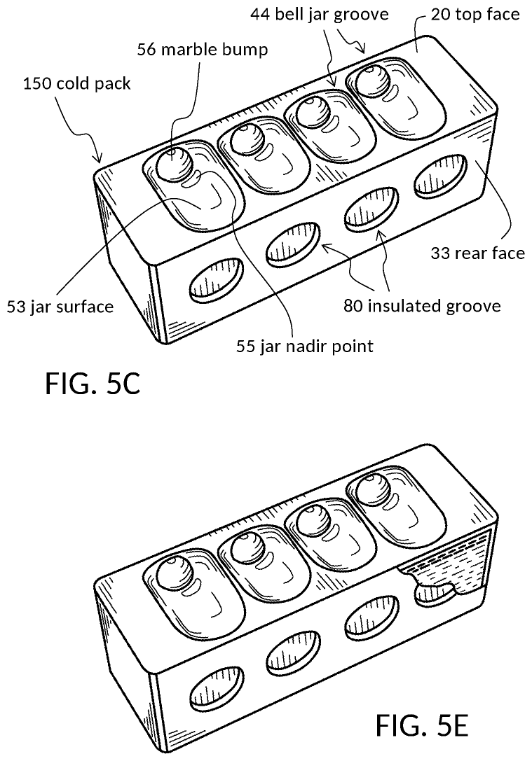

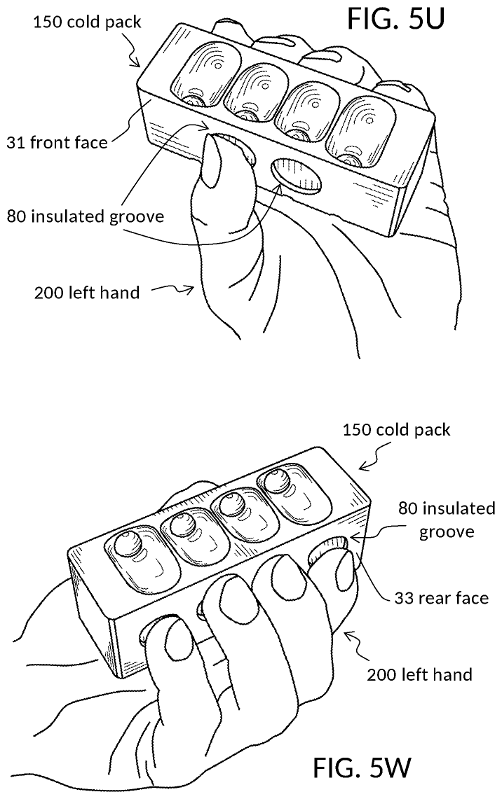

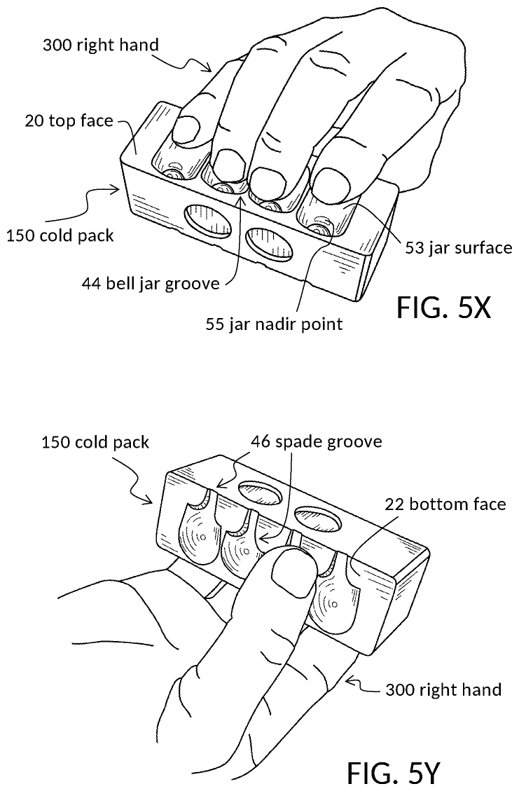

FIG. 5A illustrates the fifth embodiment of the present disclosure, nicknamed The Bar, showing a fidget cold pack 150 with freezer gel 90 in a cutaway, as an enhancement to the fidget cold pack from the second embodiment. Additional, insulated circular grooves are provided on surrounding faces, with two of these insulated grooves 80 visible on front face 31 in FIG. 5A. Otherwise similar grooves from the second embodiment are provided on the top face and the bottom face. In FIG. 5A, four bell jar grooves 44 with marble bumps 56 are visible on top face 20. Edges of four spade groove 46 on the bottom face are visible where they intersect front face 31. In the fifth embodiment, all faces outside of grooves are heat-conducting and suitable for contact with human skin. This means that any available unused flat surfaces on front face 31, right face 35, etc. can be used to apply cold therapy that bell jar grooves 44 and spade grooves 46 cannot address.

FIG. 5B illustrates bottom face 22 of fidget cold pack 150, showing four spade groove 46 on said bottom face 22, and two insulated grooves 80 on front face 31. There is a cutaway showing freezer gel 90 within the pack.

FIG. 5C illustrates rear face 33 of fidget cold pack 150, showing four insulated grooves 80 on said rear face 33. This figure also shows marble bumps 56 in bell jar grooves 44 from a new angle. The deep-cut nature of a bell jar groove can be appreciated from this view angle. As previously discussed, the nadir point 55 of a bell jar groove sinks relatively deep into it container face. In the fifth embodiment, this allows a finger to stand on its fingertip when gripping the groove from the front side. Jar surface 53 slopes up from the nadir point towards rear face 33, but bell jar groove 44 still retains an appreciable acute edge where its jar surface 53 meets the surface of top face 20. The same perspective view of fidget cold pack 150 is seen again in FIG. 5E, without reference numerals, and with a cutaway showing a freezer gel encased within the cold pack.

FIG. 5U and FIG. 5W illustrates the operation of the fifth embodiment, The Bar, when it is picked up by insulated grooves. A left hand 200 holds fidget cold pack 150 by one of the two insulated grooves 80 on front face 31, and by the four insulated grooves on the rear face, during a break in a cold therapy session. FIG. 5W continues the illustration of the same left hand 200 from FIG. 5U, showing the four fingers gripping the four insulated grooves 80 on rear face 33 of fidget cold pack 150. FIG. 5U and FIG. 5W again demonstrate the ergonomics of grooves, in particular insulated grooves in the fifth embodiment, as used by human hands with opposable thumbs.

FIG. 5X and FIG. 5Y illustrate the operation of The Bar, when it is gripped by cold grooves for cold therapy. This time a right hand is used to illustrate The Bar being picked up from the rear face. In FIG. 5X, right hand 300 treats its four fingertips and its thumb tip to cold therapy by simply gripping bell jar grooves 44 on top face 20 of fidget cold pack 150. As discussed earlier about FIG. 5C, jar surface 53 slops downward towards bell jar nadir point 55. When gripping from the rear face, the four fingers thus lay flatter on the groove surface, with a larger contact interface for a wider treatment coverage, compared to gripping the same bell jar grooves 44 from the front face, as shown in for instance FIG. 2Q with the second embodiment. FIG. 5Y shows the same right hand 300 treating fingers to cold therapy by gripping cold grooves on cold pack 150. The thumb tip grips one of four spade grooves 46 on bottom face 22.

The Bar is an example of a simple and concise fidget cold pack that still delivers all desirable outcomes mentioned at the end of the summary section. It allows users to securely grip the cold pack with just fingertips and a thumb tip. It can be effectively fidgeted in hand throughout a dynamic therapy session. Fingertips can be treated simply by gripping the cold grooves 44 and 46 as shown in FIG. 5X and FIG. 5Y. But other finger surfaces, the hand, and any other body parts can also be treated using any available surface from any container face. Fingers can take refuge during breaks with insulated grooves 80 as shown in FIG. 5U and FIG. 5W. And lastly, The Bar can be easily shifted, flipped, rotated, and otherwise manipulated by just a single hand.

### Sixth Embodiment—Tom

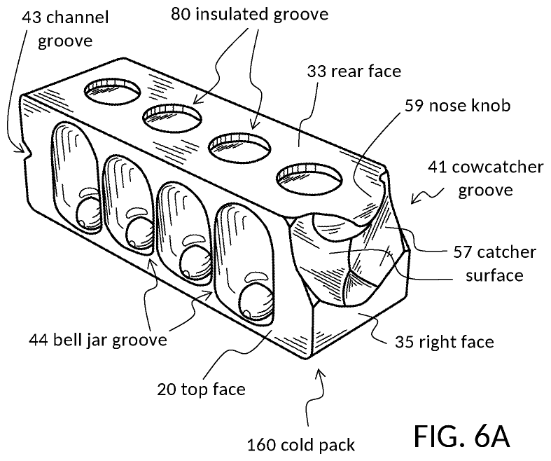

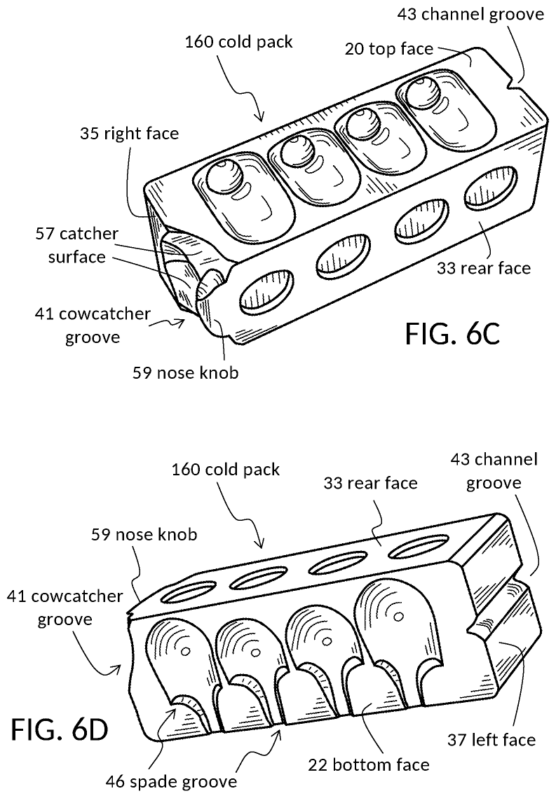

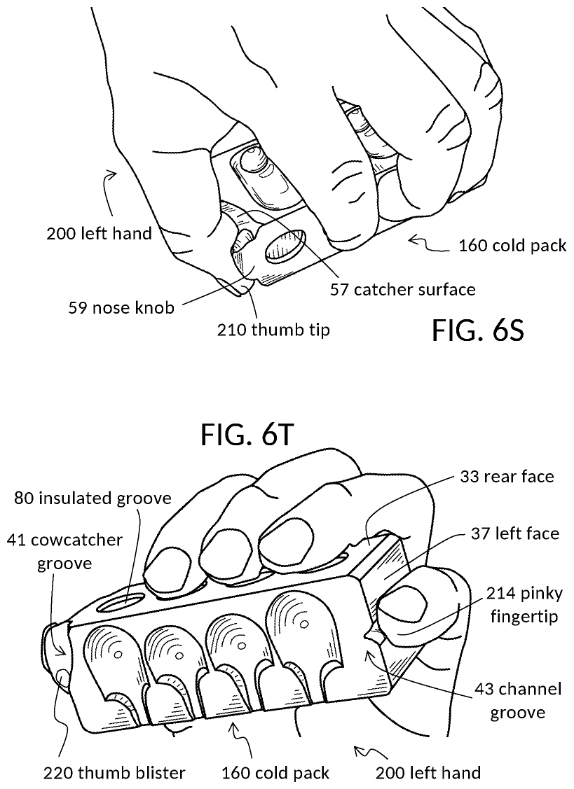

FIG. 6A illustrates the sixth embodiment of the present disclosure, nicknamed Tom, showing a fidget cold pack 160 with a cowcatcher groove 41 on right face 35, and a channel groove 43 on the left face. The rest of this cold pack 160 is otherwise inherited from cold pack 150, The Bar, with four bell jar grooves 44 visible on top face 20, and four insulated grooves 80 visible on rear face 33. Cowcatcher groove 41 resembles V-shaped pilots on locomotives that clear obstacles from train tracks. Thus the name. A cowcatcher groove 41 comprises two catcher surfaces 57, and a nose knob 59. Catcher surface 57 roughly fits a finger, like most finger grooves in the present disclosure.

One catcher surface 57 is carved deep into the surface of right face 35, at the corner where right face 35 meets top face 20 and rear face 33. The other catcher surface 57 is a mirror image carved into the corner of right face 35, rear face 33 and the bottom face. The two catcher surfaces 57 meet in the middle of right face 35, forming a V-shape. The edge where rear face 33 meets right face 35 remains uncarved, and forms Nose knob 59. One purpose of nose knob 59 is to be an obstacle for the finger, or viewed from a different perspective, to be an aid for gripping. In this way, nose knob 59 serves the same purpose for cowcatcher groove 41, as a marble bump does for a bell jar groove.

FIG. 6C is a re-orientation of the same cold pack from FIG. 6A, now showing top face 20 of cold pack 160 pointing upward, and rear face 33 pointing towards a viewer. Cowcatcher groove 41 is shown carved deeply into right face 35, comprising catcher surfaces 57 and nose knob 59. Edges of channel groove 43 can be see where it intersects top face 20.

FIG. 6D continues the illustration of Tom, now showing a clear view of channel groove 43 on left face 37 of fidget cold pack 160. Four spade grooves 46 are visible on bottom face 22. Nose knob 59 and the rest of cowcatcher groove 41 are barely visible, where they intersect rear face 33 and bottom face 22. Channel groove 43 spans one axis of left face 37, starting on the front face, and ending on bottom face 22. In this embodiment, it is a channel with a v-shaped cross section cut into left face 37, about ⅓ of the width of a finger. One purpose of channel groove 43 is to act as a gripping aid.

Tom is derived from The Bar, the fifth embodiment. And The Bar is derived from the second embodiment. Because Tom retains the same features of The Bar, Tom can be held and fidgeted by fingers and a thumb using the same postures as demonstrated previously in FIG. 2Q, FIG. 2R, FIG. 2S, FIG. 5U, FIG. 5W, FIG. 5X, and FIG. 5Y. Presently, FIG. 6S and FIG. 6T demonstrate yet more way hold and fidget the sixth embodiment. They illustrate the operation of the sixth embodiment, or Tom, when gripped by the thumb, and the pinky in opposition to each other. This is a different way to grip a cold pack, compared to previous embodiments.

FIG. 6S shows left hand 200 treating thumb tip 210 with a catcher surface 57 of fidget cold pack 160. Nose knob 59 serves as both gripping aid and as a guard rail to keep the thumb in place. The thumb pushes down on catcher surface 57 against the pinky which is holding the other end of the pack, and is out of sight in FIG. 6S. However, this is made clear in FIG. 6T where pinky fingertip 214 is shown gripping channel groove 43 on left face 37. Cold pack 160 is clearly seen as primarily caged in by the opposable thumb and the pinky of left hand 200. And the rest three fingers rest on insulated grooves 80 on rear face 33.

The sixth embodiment shows that surrounding faces can also provide finger grooves for cold therapy, not just insulated grooves. The cowcatcher groove is a deeper cut than previously-discussed grooves. The cowcatcher groove, together with the channel groove on Tom, allows additional surfaces of the thumb to be treated with the cold pack, including thumb blister area 220 pinned against cowcatcher groove 41 in FIG. 6T. Not only can the radial side of the thumb be treated this way, any other one of the four fingers can also be treated with the cowcatcher groove, by simply having the thumb grip the channel groove instead, in opposition to the cowcatcher groove. With Tom, all six faces of The Bar are now utilized for cold therapy fidgeting.

### Seventh Embodiment—the Hat

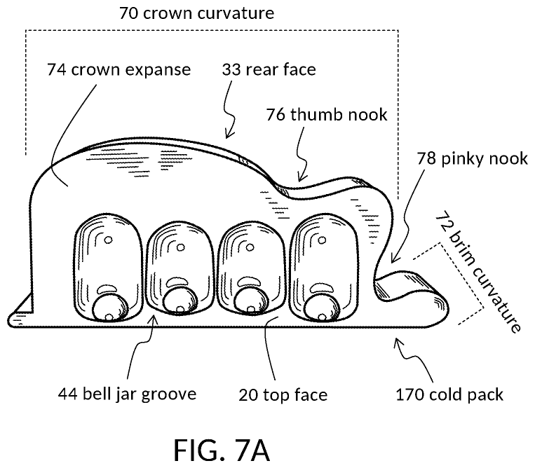

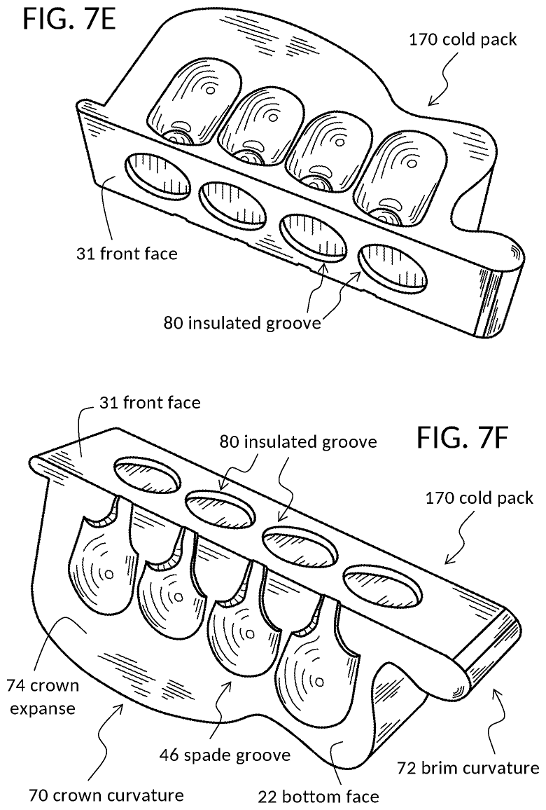

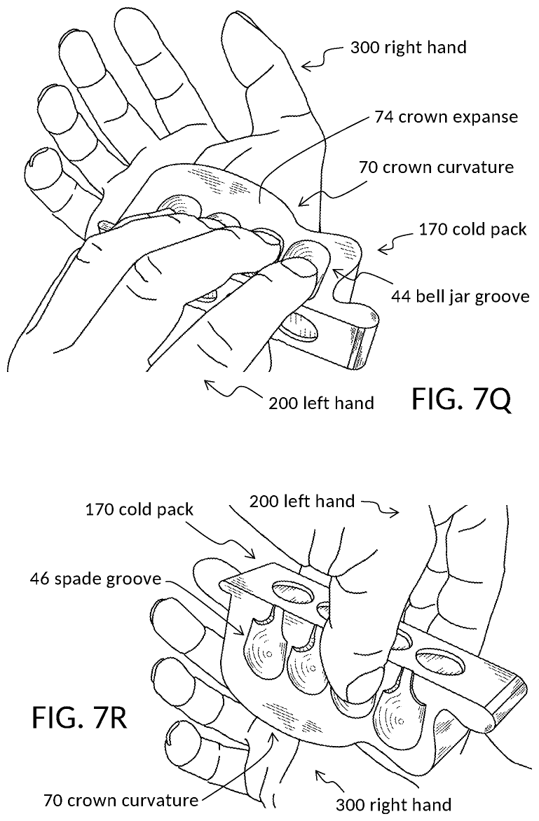

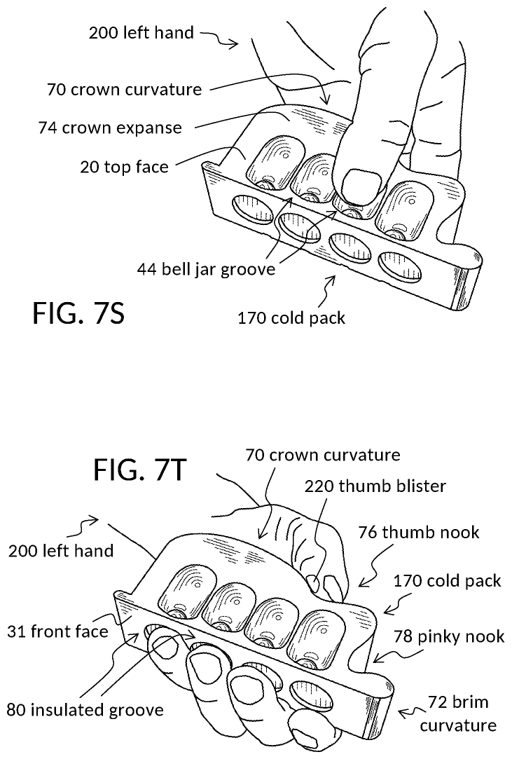

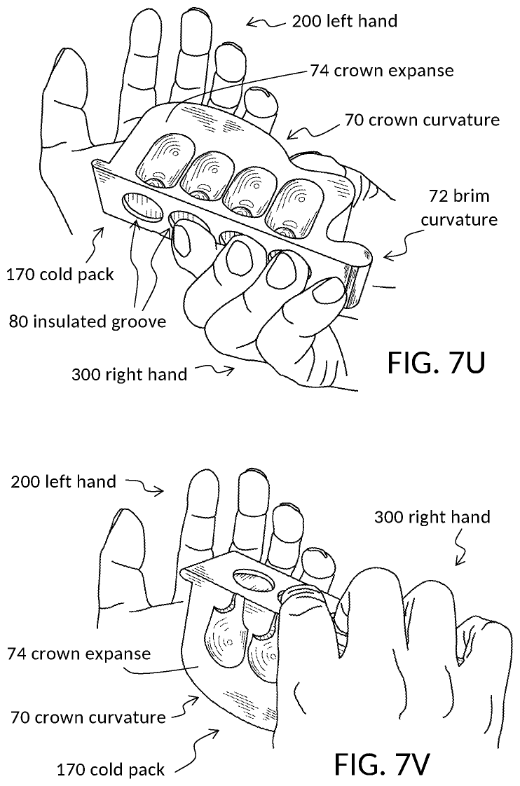

FIG. 7A illustrates the seventh embodiment of the present disclosure, nicknamed The Hat, showing a fidget cold pack 170 where a top face 20 has a profile of a not-so-tall tall hat, or as some may claim, a well-fed boa constrictor. The Hat demonstrates that surrounding faces of a pack can be shaped into curves of various radii, and made with a mildly heat-conducting material suitable for contact with human skin, such that these curved surfaces may be used to treat a finger, another hand, or other body parts. In fact, there are no distinct surrounding faces. These surrounding faces merge into one long continuous surface in the seventh embodiment. For the purpose of this specification, we'll call continuous curved surface that form the crown part of The Hat the crown curvature 70. A part of crown curvature 70 covers the equivalent of rear face of 33 of The Bar, and is labeled symbolically in FIG. 7A. The brim surface shown on the right in FIG. 7A will be called brim curvature 72. There are two concave sections in the continuous curved surface. The first is the thumb nook 76 next to the bell jar groove 44 closest to brim curvature 72. The second is the pinky nook 78 between crown curvature 70 and brim curvature 72.

FIG. 7A also illustrates that The Hat inherits some characteristics of The Bar, namely four bell jar grooves 44 on top face 20, and four spade grooves on the bottom face which are not visible in FIG. 7A. The Hat has an expanded volume, similar to The Brick, providing a longer-lasting therapy. But The Hat does not provide a second row of grooves. Some of this expanded space is presented as crown expanse 74 with a flat and heat-conducting surface surrounded by crown curvature 70, and can be used for cold treatments.

FIG. 7E and FIG. 7F continue the illustration of The Hat. FIG. 7E shows four insulated grooves 80 on its front face 31. These are the same insulated grooves as found on The Bar, but the row of four grooves are provided on front face 31 of cold pack 170, and not on the rear face. FIG. 7F shows the same four insulated grooves 80 on the front face 31 of cold pack 170, plus another four spade grooves 46 on bottom face 22. These four spade grooves are provided in the same way they are configured on The Bar. Crown expanse 74 exists on bottom face 22 as well. And the profile of crown curvature 70 and brim curvature 72 can be observed in this figure, too. In the seventh embodiment, these expanded spaces and curved surfaces are semi-rigid and retain their shape hot or cold. In other embodiments, similar expanded spaces and surfaces may be made of flexible materials such that they can be molded to the contour of body surfaces on demand.

FIG. 7Q and FIG. 7R illustrate the operation of the seventh embodiment, where the same cold pack 170 is used to treat fingertips of left hand 200, and at the same time the palm surface of right hand 300. Left hand 200 simply grips the pack via bell jar grooves 44 for treatment to fingertips (shown in FIG. 7Q), and via a spade groove (shown in FIG. 7R) for treatment to the thumb tip. And at the same time, the same left fingers and left thumb direct cold pack 170 to treat the palm surface of right hand 300 via crown expanse 74 and crown curvature 70. The way left hand 200 uses the seventh embodiment (The Hat) is identical to how a left hand would use the fifth embodiment (The Bar). The expanded spaces and curved surfaces on The Hat, however, makes it more versatile in treating additional body parts such as right hand 300, as shown in FIG. 7Q and FIG. 7R.

FIG. 7S illustrate another aspect where The Hat is used in the same way as The Bar. Left hand 200 grips bell jar groove 44 on top face 20 of cold pack 170, from the side of crown curvature 70. The crown curvature is the equivalent of the rear face of The Bar. The crown expanse 74 is not so large as to obstruct left hand 200 in holding The Hat this way.

FIG. 7T illustrate yet another aspect where The Hat is used similar to how The Bar is used during one of many breaks in a dynamic cold therapy session. Left hand 200 holds cold pack 170 with four fingers on insulated grooves 80 on front face 31, and the thumb tip on crown curvature 70.

FIG. 7T also illustrates a use of thumb nook 76. In other variations of The Hat, thumb nook 76 in crown curvature 70 may be made heat-insulating. Or an insulated groove may be provided there. But in this seventh embodiment, thumb nook 76 is heat-conducting which allows thumb blister area 220 of a cellist to seek cold treatment thereby. As mentioned before, the radial side of a cellist's left thumb is a sore spot prone to developing blisters after playing thumb positions. Ample surface area in the nook can treat thumb tips of all sizes. Pinky nook 78 operates in a similar fashion and can be used for smaller fingers such as the pinky. Brim curvature 72 can be used for spot treatments.

FIG. 7U and FIG. 7V illustrate an alternative holding posture with fidget cold pack 170. This time right hand 300 holds the cold pack, reaching from the side where brim curvature 72 is. Only three fingers grip insulated grooves 80. This posture points crown expanse 74 and a large part of crown curvature 70 outward from right hand 300, thus making it easier to treat large areas of other body parts, such as the palm of left hand 200.

### Eighth Embodiment

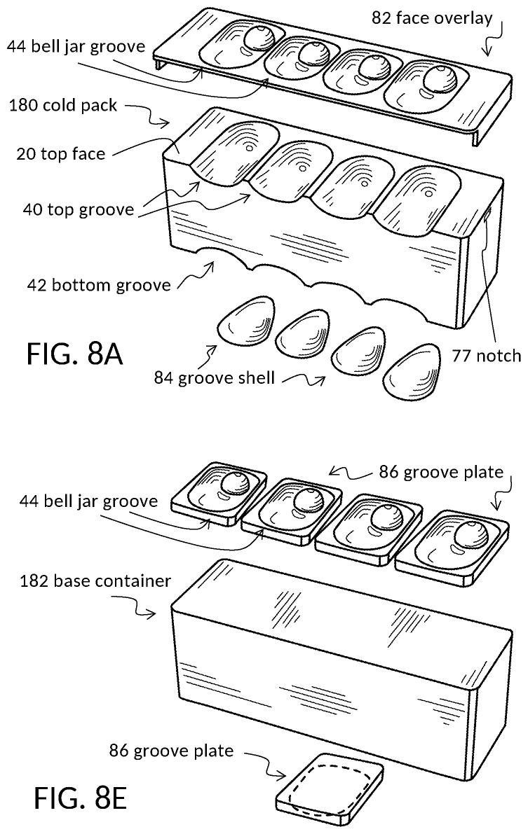

FIG. 8A illustrates one aspect of the eighth embodiment, showing how removable face overlays and groove shells can be used with a version of the first embodiment. Fidget cold pack 180 is usable as a basic, standalone cold pack. It is equipped with four top grooves 40 on top face 20, and four bottom grooves 42 on its bottom face. An attachable and removable face overlay 82 can be snapped onto top face 20, and latched onto notch 77. The overlay is provided with four bell jar grooves 44. Various materials can be used to make overlay 82, including flexible materials, heat-conducting or heat-insulating, textured, grippy, etc. Various types of grooves may be provided, instead of bell jar grooves 44 depicted. This allows the basic cold pack 180 to be thus customized for desired tactile feedback based on individual needs. Individual groove shells 84 can be made similarly with different material types, and furnished with different groove textures, bumps and guard rails. These groove shells can be attached to bottom grooves 42 via adhesives or hook-and-loop fasteners.

FIG. 8E illustrates another aspect of the eighth embodiment, showing a plain rectangular bar serving as a rigid base container 182 that can be customized with individual groove plates 86 with bell jar grooves 44 on any of its six faces. Each groove plate 86 can be made of different materials and furnished with a custom groove pattern, similar to face overlays and groove shells described earlier. Similar to groove shells, groove plates can be attached to base container 182 via adhesives or hook-and-loop fasteners. This gives complete freedom to end users for customizations of groove placement that fit individual needs. A base container 182 together with attached groove plates 86 thus form a complete fidget cold pack.

### Ninth Embodiment—Kidney

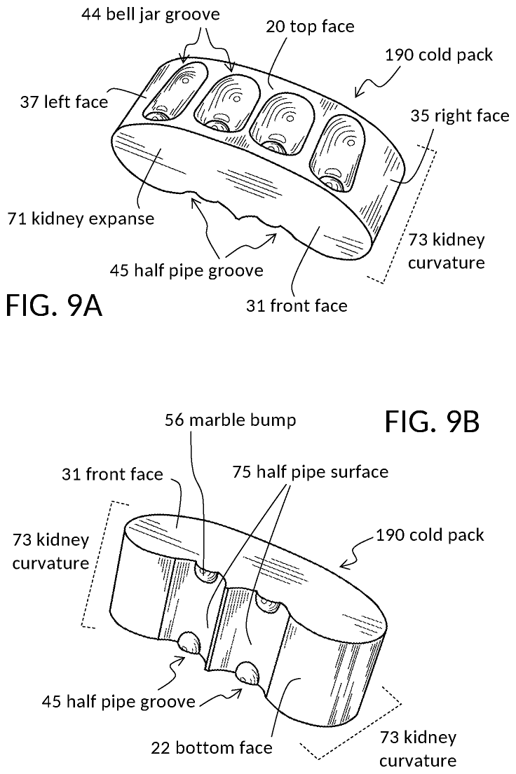

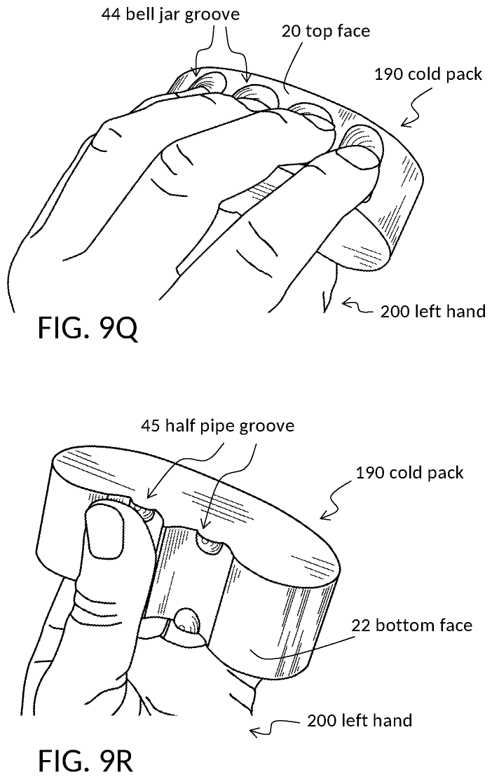

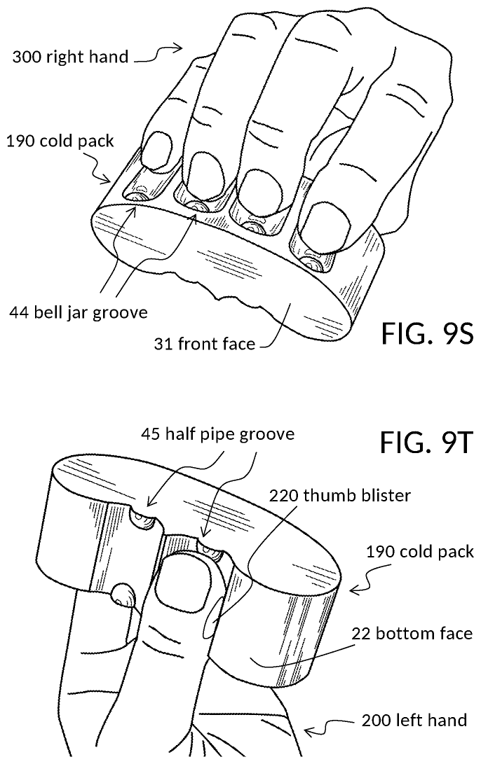

FIG. 9A illustrates the ninth embodiment of the present disclosure, nicknamed Kidney, showing a fidget cold pack 190 where four bell jar grooves 44 are arranged on a curved top face 20, matching the natural contour of fingertips in a relaxed hand posture as shown in FIG. 1G. Kidney demonstrates a principle described earlier in the present disclosure, that container faces may be flat or curved, and that boundaries between faces are not always explicitly demarcated with geometric features. For instance, the ninth embodiment comprises two kidney curvature 73 portions, one on each end of the cold pack. These are roughly equivalent to the left face and right face of a basic cold pack 100 shown in FIG. 1A. On Kidney, however, the two kidney curvatures 73 merge into the curved top face 20 without any geometric boundaries. Nevertheless, one skilled in the art will still be able to make reasonable calls marking crude boundaries between them. One would draw a boundary line between the bulk of one end of the pack, and the closest bell jar groove 44, to call out right face 35 and left face 37. Kidney also provides a generous kidney expanse 71 for various types of cold treatment to a large body surface. In this embodiment, kidney expanse 71 takes the entirety of front face 31. Outlines of two half pipe grooves 45 are visible where they intersect front face 31.

FIG. 9B continues the illustration of Kidney, showing two large half pipe grooves 45 on bottom face 22 of cold pack 190. The two ends of bottom face 22 of this cold pack are taken up by the two kidney curvatures 73 that flank it. The middle of bottom face 22 are carved to form two half pipe grooves 45. Each half pipe groove 45 comprises a generous half pipe surface 75, flanked from the front face 31 and the rear face each with a marble bump 56.

Kidney shares many similar features found in The Hat. It has ample flat surfaces and curved surfaces. These surfaces are made with a mildly heat-conducting material suitable for contact with human skin, just like The Hat. Thus, Kidney can also be used by one hand to treat a second body part. But Kidney provides flat surfaces on its surrounding faces, such as the front face and the rear face, whereas The Hat provides flat expanses on its top face and bottom face. Kidney provides curved surfaces on its top face and bottom face, whereas The Hat provides curved surfaces mainly on its rear face.

FIG. 9Q and FIG. 9R illustrate self-treatment operations of the ninth embodiment, where four fingers of left hand 200 grip bell jar grooves 44 on top face 20 of fidget cold pack 190, and the thumb grips one of the two half pipe grooves 45 on bottom face 22. Despite differences in look, Kidney is held the same way The Hat and The Bar are held. Thus, FIG. 9Q can be compared to FIG. 2Q, and FIG. 9R can be compared to FIG. 2R.

FIG. 9S illustrate a right hand 300 gripping bell jar grooves 44, reaching from the rear face, instead of from front face 31 of cold pack 190. As mentioned in the discussion about FIG. 5X showing The Bar, this approach from the rear face presents a larger treatment groove surface to a finger that lays flatter on the groove surface.

FIG. 9T illustrates left hand 200 holding cold pack 190, with the thumb gripping another one of the two half pipe grooves 45 on bottom face 22. Compare FIG. 9T to FIG. 9R to see the difference in thumb tip surfaces being treated between these two grips. The finger posture in FIG. 9T allows the thumb to wedge its thumb blister 220 area firmly into the groove surface, providing a better treatment option for blistering issues of a cellist's thumb.

### Tenth Embodiment—Rowboat

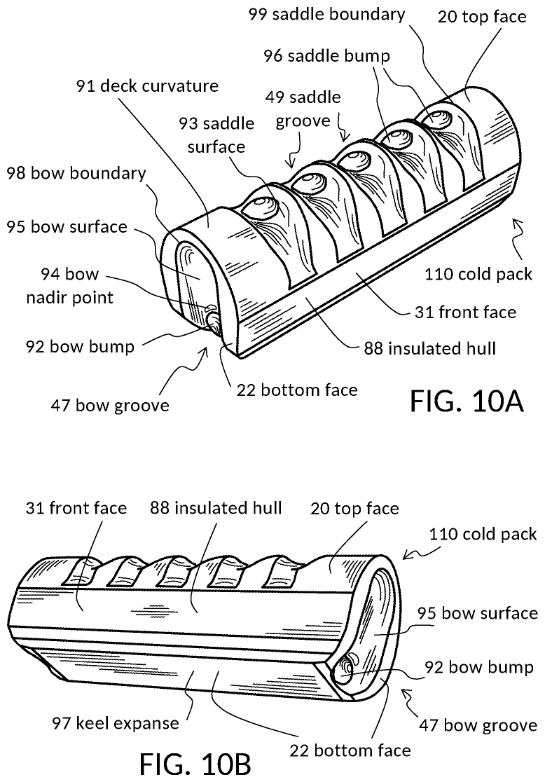

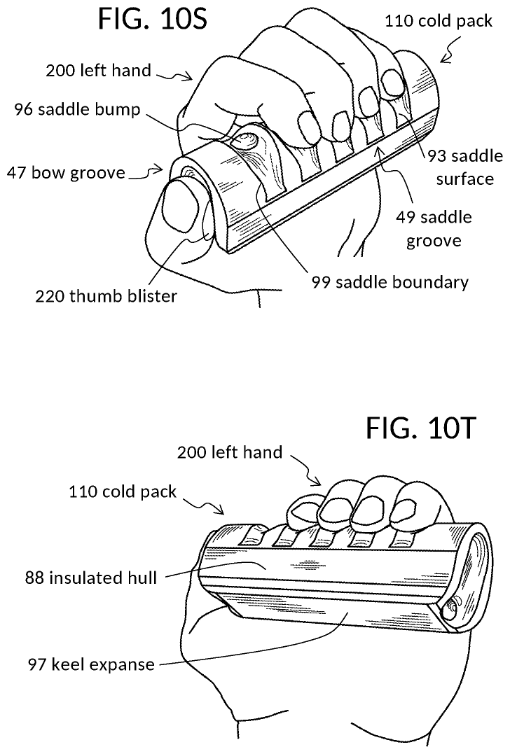

FIG. 10A illustrates the tenth embodiment of the present disclosure, nicknamed Rowboat, showing a fidget cold pack 110 where five saddle grooves 49 are provided on a curved top face 20 which follows the inner contour of four fingers as they curl into a clenched fist. An insulated hull 88 strip runs the entire length of the pack on front face 31. And a bow groove 47 is provided on the up-sweeping left end of curved bottom face 22. Each saddle groove 49 is provided with a saddle surface 93 which flanks both front and rear sides of the curved top face 20, and meets the surface of top face 20 at an acute angle at saddle boundary 99. A saddle bump 96 is situated in the center of each saddle groove 49. The bow groove 47 is very similar to a bell jar groove, but a bow groove 47 is longer and wider, with its bow surface 95 meeting the surface of bottom face 22 at an acute angle at a majority of bow boundary 98. Bow groove 47 is provided with a bow bump 92 next to its row nadir point 94, similar to the ball bump in a bell jar groove. But a bow groove 47 is not terminated with a wall at the bow bump 92 end of its bow surface 95. Instead, bow surface 95 curves and joins with the rest of bottom face 22, as will be further discussed. The two ends of curved top face 20 become deck curvatures 91, one on the left end of top face 20, and the other on the right end.

FIG. 10B illustrates parts of bottom face 22 on fidget cold pack 110 that were not visible in FIG. 10A. Note that this figure shows the same front face 31, insulated hull 88, and top face 20 as FIG. 10A. In FIG. 10B, keel expanse 97 curves up at the right end to form a second bow groove 47. For the purpose of describing Rowboat, the tenth embodiment, I do not make a distinction between bow and stern, as the Rowboat is symmetrical—the right end and the left end are identical. This view clearly demonstrates the curved nature of bottom face 22, and how bow surface 95 flows out of bow groove 47's boundary, into keel expanse 97. Bow bump 92 is visible as a hemisphere situated in the middle of this flow from bow surface 95 into keel expanse 97. On Rowboat, surrounding faces include only front face 31, and the rear face on the opposite side. There are only four named faces on Rowboat: front, rear, top and bottom.

Rowboat demonstrates similar principles discussed earlier with Kidney, that container faces may be flat or curved, and that boundaries between faces are not always explicitly demarcated with geometric features. Rowboat demonstrates a related principle of the latter, that is, geometric features do not necessarily demarcate container faces. In FIG. 10B, there is a significant bending of surface between keel expanse 97 and bow surface 95, but it does not constitute a boundary of two faces. As will be described in the operations section, the opposable thumb movement of a hand is still at play, when four fingers pin the body of the pack from top face 22 against a thumb pushing back on bow surface 95 of bottom face 22.

FIG. 10S illustrates one self-treatment operation of the tenth embodiment, where four fingers of left hand 200 grab four out of five available saddle grooves 49 on cold pack 110. These four fingers curl into a clenched-fist posture, and grip saddle grooves by saddle bumps 96. Fingertips pin saddle surface 93 against the palm, thus securing Rowboat in a hand. All aspects of the thumb and especially the thumb tip including thumb blister area 220 prone to blisters in cellists can be treated with the large surface of bow groove 47. The force generated by the thumb pushing on bow groove 47 is countered by the four fingers bracing against saddle boundary 99.

FIG. 10T shows another perspective of the same left hand 200 self-treating with Rowboat from FIG. 10S. The palm is seen bracing keel expanse 97 and the counterpart of the visible insulated hull 88 on the opposite side, against a clenched fist. The finger dynamics shown here is different from that used with Tom, the sixth embodiment, where the pinky caps the opposite end of a pack from the thumb. Rowboat is primarily held by the palm and the four fingers. The keel expanse 97 of fidget cold pack 110 can be used to treat the inside surface of the palm, if needed. The pack can be rolled a little such that the expanse is touching the flesh of the palm. This embodiment can be used to treat blistering issues arising from the use of mallets by marimba players. It also helps treat blistering issues from sports injuries in baseball, golf, rowing, etc. In addition, minor burns on the hand is a common injury in kitchens. This embodiment is also helpful in preventing blistering, when applied right after a minor incident.

The thumb can be treated with either thumb groove on Rowboat. Due to the ergonomics of how human hands work, five finger grooves are provided on Rowboat, instead of the typical set of four grooves in most embodiments. A typical adult hand will need some space between the thumb groove and the index finger, when holding Rowboat. Alternatively, Rowboat can be gripped with only the thumb on one bow groove, and another finger on the opposite bow groove.

### Alternative Embodiments

While the present disclosure is susceptible to further modifications and variations, a selection of embodiments is shown in drawings and described in the specification. It is understood that the selection does not set a limit on the disclosure. On the contrary, the intention is to cover all modifications, alternative constructions, combinations, and equivalents found within the spirit and scope of the present disclosure.

A person skilled in the art will understand that further changes can be made to embodiments described, and still achieve desired results. It is also apparent that different combination of features described in selected embodiments can produce similar advantages and benefits. In some cases, a subset of these features can still produce the same advantages and benefits, and may even be desirable in certain use cases. All of above are a part of the present disclosure.

### Conclusion

Embodiments presented in this disclosure address an unrecognized problem where a cold pack can be held and fidgeted by the same fingertips that require dynamic cold therapy treatments, in order to prevent formation of blisters caused by repeated pressure and friction. Fidgetable cold packs described in this disclosure address this unrecognized problem by providing a rigid portion with heat-conducting grooves and heat-insulating surfaces, and by furnishing these grooves and surfaces with acute edges, jutting bumps, guard rails and other textures to provide grip.

In contrast to traditional rigid food-cooling packs and flexible body-treatment packs, fidget cold packs presented in this disclosure achieve following desirable outcomes:
1. A user can securely grip a thermal pack with just fingertips and a thumb tip.
2. A user can actively fidget a pack in hand throughout a dynamic therapy session.
3. Fingertips, the hand, and any other surfaces of the body can be treated.
4. Fingers can take refuge when holding the pack outside of active treatment.
5. A pack can be shifted, flipped, rotated, and otherwise manipulated by a single hand.

1. A thermal pack to be fidgeted by a human hand, comprising:
a waterproof container;
said container encasing a heat-transferring substance;
said container having in general a plurality of container faces;
said container faces comprising in general a top face, a bottom face, and one or more surrounding faces;
said top face and said bottom face being substantially rigid;
said top face being connected to said bottom face in a substantially rigid manner;
said surrounding faces border said top face and said bottom face;
said surrounding faces comprising in general a front face and a rear face;
said container faces providing and hosting a plurality of finger grooves;
a plurality of said finger grooves being top grooves on said top face;
one or more of said finger grooves being bottom grooves on said bottom face;
each said finger groove having a groove surface demarcated from its hosting container face by a groove boundary;
each said groove surface having a groove nadir point of a depth greater than 3 mm from the average boundary height of said groove boundary;
each said groove surface being shaped to receive the skin of a fingertip or a thumb tip; and
said groove nadir point of each said top groove being separated from said groove nadir point of the nearest top groove for no more than 40 mm.

2. The thermal pack of claim 1 wherein said heat-transferring substance is a gel or liquid capable of being heated or chilled, with a volume less than a sphere of 70 mm radius.

3. The thermal pack of claim 1 wherein said heat-transferring substance is created on demand from an endothermic chemical reaction of a plurality of ingredients.

4. The thermal pack of claim 1 wherein each said finger groove is constructed from materials with at least one these properties: heat-insulating, heat-conducting, textured, and grippy.

5. The thermal pack of claim 1 wherein each said finger groove is provided with optional groove shells, plates or overlays, constructed from materials with at least one these properties: flexible, heat-insulating, heat-conducting, textured, and grippy.

6. The thermal pack of claim 1 wherein at least one of said surrounding faces is constructed from materials with at least one these properties: flexible, heat-insulating, heat-conducting, textured and grippy.

7. The thermal pack of claim 1 wherein at least one of said finger grooves has at least a portion of its groove surface meet its hosting container face at a near acute angle.

8. The thermal pack of claim 1 wherein at least one of said finger grooves is provided on its surface with at least one of raised ridges, indented valleys, jutting bumps, and guard rails, on its groove surface.

9. The thermal pack of claim 1 wherein said surrounding faces form a curved contour.

10. The thermal pack of claim 1 wherein said top face is a curved surface.

11. The thermal pack of claim 1 wherein said bottom face is a curved surface.

12. The thermal pack of claim 1 further comprising:
one or more of said container faces providing and hosting one or more rows;
each of said rows hosting a plurality of said finger grooves known as row grooves, and bordering one of said surrounding faces known as its border face;
said row grooves forming a curve along said border face, with their groove nadir points being located no more than 30 mm away from said border face; and
one of said rows being on said top face bordering said front face.

13. The thermal pack of claim 12 further comprising:
one of said rows being on said top face bordering said rear face.

14. The thermal pack of claim 12 further comprising:
a plurality of said finger grooves being bottom grooves on said bottom face;
said groove nadir point of each said bottom groove being separated from said groove nadir point of the nearest bottom groove for no more than 40 mm; and
one of said rows being on said bottom face bordering said front face.

15. The thermal pack of claim 14 further comprising:
four of said top grooves being heat-conducting and located on said top face;
four of said bottom grooves being heat-conducting and located on said bottom face;
two of said finger grooves being heat-insulating and located on said front face; and
four of said finger grooves being heat-insulating and located on said rear face.

16. The thermal pack of claim 1 wherein each said groove surface has at least an area of π*5 mm\hat{ }2.

17. The thermal pack of claim 1 wherein at least one of said finger grooves has a bell jar-shaped groove surface, with a marble-shaped bump protruding at the bottom end of said bell jar shape.

18. The thermal pack of claim 1 wherein at least one of said finger grooves has a spade-shaped groove surface, with guardrails on its hosting container face flanking the handle end of said spade shape.
